(19) United States
(12) Patent Application Publication
(10) Pub. No.: US 2008/0248067 A1
(43) Pub. Date: Oct. 9, 2008

(54) Immunomodulating Compositions and Uses Therefor

(75) Inventor: Ian Hector Frazer, St. Lucia (AU)

Correspondence Address: SEED INTELLECTUAL PROPERTY LAW GROUP PLLC, 701 FIFTH AVE, SUITE 5400, SEATTLE, WA, 98104, US

(73) Assignee: THE UNIVERSITY OF QUEENSLAND, St. Lucia (AU)

(21) Appl. No.: 11/911,507

(22) PCT Filed: Apr. 18, 2006
(86) PCT No.: PCT/AU2006/000514

§ 371 Date: Jun. 16, 2008

(30) Foreign Application Priority Data
Apr. 14, 2005 (AU) 2005901872

(51) Int. Cl. A61K 39/00; A61P 37/02

(52) U.S. Cl. 424/277.1; 424/184.1

(57) ABSTRACT

The present invention discloses the use of an inhibitor of IL-10 function and an immune stimulator that stimulates the priming of an immune response to a target antigen, in methods and compositions for stimulating and prolonging host immune responses to the target antigen. The methods and compositions of the present invention are particularly useful in the treatment or prophylaxis of a range of conditions including pathogenic infections and cancers.

### FIELD OF THE INVENTION

This invention relates generally to methods and agents for modulating immune responses. More particularly, the present invention relates to the use of an inhibitor of IL-10 function and an immune stimulator that stimulates the priming of an immune response to a target antigen, in methods and compositions for stimulating and prolonging host immune responses to the target antigen. The methods and compositions of the present invention are particularly useful in the treatment or prophylaxis of a range of conditions including pathogenic infections and cancers.

### BACKGROUND OF THE INVENTION

“Original antigenic sin” is a term coined to recognize that when a host is sequentially stimulated with two cross-reacting antigens, the induced immune responses will be directed only toward to the first antigen, and was first described for the antibody responses during influenza infection (Webster, 1966, J. Immunol. 97:177-183). Although some level of cross-reactivity with emergent viruses may explain persistence of the seminal antibodies, the mechanism which stops the immune system of the infected host from producing high affinity neutralizing antibodies against emergent viral variants is not clear.

In the case of HIV infection, original antigenic sin manifests as B cell clonal dominance, where the original responding B cells are “locked in” through a process termed “deceptive imprinting” or a “repertoire freeze” (Kohler et al., 1994, Immunol. Today 15:475-478). This clonal dominance involves restricted diversity in the set of antibodies produced against HIV and apparently can weaken the adaptation of the immune response to emerging mutants and favor viral persistence.

Original antigenic sin has also been observed for CTL responses (Klenerman and Zinkernagel, 1998, Nature 394(6692):482-485), as mice primed with one strain of LCMV respond to a subsequent infection by a CTL epitope variant strain with a CTL response directed against the initial epitope rather than against the new variant epitope.

Pre-existing T cells or antibody, cross reactive with and therefore capable of removing the variant antigen, or antigen presenting cells presenting the novel antigenic epitopes, have each been considered factors contributing to inhibition of development of responses to the novel epitopes of the variant antigen (Da Silva, 2001, Virology 290(2):350-60; McMichael, 1998, Nature 394(6692):421-422). Recently, Liu et al. (2003, J. Immunol. 171:4765-4772) found that prior immunity to a papillomavirus (PV) capsid protein inhibits induction from naïve CD8⁺ T cells of an IFN-γ response to a MHC class I restricted epitope linked to the capsid protein, following immunization with a capsid expressing the class I restricted epitope. Additionally, Liu et al. observed that this reduced response requires IL-10 production, is local to the site of PV capsid delivery, and is independent of antibody to the capsid. While concluding that these observations fit the description of original antigenic sin, these investigators proposed that local inhibition of IL-10 may be a key determinant of overcoming prior immunity to a carrier antigen when it is desired to induce a novel immune response through vaccination to a viral or tumor antigen.

In work leading up to the present invention, it was discovered that PV capsid protein L1 VLP immunization generates antigen specific IL-10 secreting CD4⁺ T cells, which are required for the inhibition of subsequent antigen specific IFN-γ secretion by CD8⁺ T cells in a VLP primed host. The present inventors also discovered that VLP specific CD4⁺ T cells secrete IL-10 upon contact with dendritic cells and that the CD4⁺ T cell-educated dendritic cells favor induction of CD8⁺ T cells that secrete IL-5 but no IFN-γ. Additionally, it was found that temporary neutralization of IL-10 either in vitro or in vivo allows restoration of the ability to mount a CD8⁺ IFN-γ response to the VLP following appropriate immunization.

The above discoveries have been reduced to practice in novel methods and compositions for stimulating more efficacious immune responses against a variety of pathogenic agents, especially those that can persist and lead to chronic disease.

### SUMMARY OF THE INVENTION

Accordingly, in one aspect, the present invention provides compositions for stimulating an immune response against a target antigen in a subject that is naïve to the target antigen or that has previously raised an immune response to the target antigen. These compositions generally comprise an immune stimulator that stimulates or otherwise enhances an immune response to the target antigen and an inhibitor of IL-10 function. In some embodiments, the immune stimulator is selected from an antigen that corresponds to at least a portion of the target antigen, an antigen-binding molecule that is immuno-interactive with the target antigen and an immune-stimulating cell that stimulates or otherwise enhances an immune response to the target antigen. The target antigen is typically associated with a disease or condition of interest. In some embodiments, the target antigen is produced by a pathogenic organism or a cancer. The antigen that corresponds to at least a portion of the target antigen may be in soluble form (e.g., a peptide or polypeptide or a construct from which any one of these is expressible). Alternatively, the antigen may be a particle or cell (e.g., a virus, bacterium or whole cell) or presented by an antigen-presenting cell (e.g., a professional or facultative antigen-presenting cell). In embodiments in which the immune-modulating agent is an antigen-binding molecule, such a molecule will typically bind to or otherwise interact with the target antigen so as to reduce its level or functional activity (e.g., a neutralizing antibody). Exemplary immune-stimulating cells that may be used in concert with the inhibitor of IL-10 function are immune effector cells, including antigen-specific T lymphocytes such as but not limited to cytolytic T lymphocytes and helper T lymphocytes, T regulatory cells and B lymphocytes. In some embodiments, the composition comprises more than one immune stimulator, e.g., 2, 3, 4, 5 or more immune stimulators, which stimulate or otherwise enhance an immune response to the target antigen or to a plurality of target antigens.

Suitably, in embodiments in which the subject has previously raised an immune response to the target antigen and the immune stimulator comprises an antigen that corresponds to at least a portion of the target antigen, the amino acid sequence of the corresponding antigen is the same as the amino acid sequence of the at least said portion. In other embodiments in which the subject has previously raised an immune response to the target antigen and the immune stimulator comprises an antigen that corresponds to at least a portion of the target antigen, the amino acid sequence of the corresponding antigen is distinguished from amino acid sequence of the at least said portion by the addition, deletion or substitution of at least one amino acid residue. In illustrative examples of these embodiments, the corresponding antigen is a naturally-occurring antigen to which the subject has already raised an immune response.

In some embodiments, the inhibitor of IL-10 function is selected from soluble or defective IL-10 receptors or fragments thereof, cells expressing IL-10 receptors or fragments thereof, antigen-binding molecules that are immuno-interactive with IL-10 or an IL-10 receptor, nucleic acids that inhibit the expression of an IL-10 gene or the functional activity of an IL-10 expression product (e.g., antisense molecules, ribozymes or RNAi molecules with specificity to an IL-10 gene or its transcripts) or small molecule inhibitors of IL-10.

In some embodiments, the compositions further comprise a pharmaceutically acceptable carrier or diluent. In certain embodiments, the compositions further comprise an adjuvant that enhances the effectiveness of the immune stimulation. Suitably, the adjuvant delivers the antigen to the class I major histocompatibility (MHC) pathway. For example, such adjuvants include, but are not limited to, saponin-containing compounds (e.g., ISCOMs) and cytolysins, which mediate delivery of antigens to the cytosol of a target cell. The cytolysin may be linked to, or otherwise associated with, the antigen. In some embodiments, the cytolysin mediates transfer of the antigens from the vacuole (e.g., phagosome or endosome) to the cytosol of an antigen-presenting cell and in illustrative examples of this type, the cytolysin is a listeriolysin.

Another aspect of the present invention provides methods for stimulating an immune response in a subject that is naïve to a target antigen or that has previously raised an immune response to the target antigen. These methods generally comprise administering to the subject an effective amount of an immune stimulator and an inhibitor of IL-10 function, as broadly described above. The active components of the composition may be administered sequentially, separately or simultaneously. In certain embodiments, the immune response is a T-cell mediated immune response. Advantageously, these methods are useful for the treatment or prophylaxis of a disease or condition associated with the presence or aberrant expression of a target antigen in a subject. In certain embodiments, the disease or condition is selected from a pathogenic infection, a disease characterized by immunodeficiency or a cancer. In accordance with the present invention, the inhibitor of IL-10 function will inhibit the production of IL-10 that would otherwise be produced in the absence of the inhibitor, which will thereby maintain a subject's capacity to mount a CD8⁺ IFN-γ response to the target antigen(s) following subsequent delivery of the immune stimulator and optionally the inhibitor of IL-10 function. Accordingly, in some embodiments, the methods of the invention further comprise administering at least one other effective amount of the immune stimulator and optionally at least one other effective amount of the inhibitor of L-10 function, to thereby maintain or enhance the immune response to the target antigen(s).

In yet another aspect, the invention contemplates the use of an inhibitor of IL-10 function and an immune-stimulating agent as broadly defined above in the manufacture of a medicament for stimulating or enhancing an immune response to a target antigen.

In still another aspect, the invention resides in the use of an inhibitor of IL-10 function and an immune-stimulating agent as broadly defined above in the manufacture of a medicament for treating or preventing a disease or condition associated with the presence or aberrant expression of a target antigen.

### BRIEF DESCRIPTION OF THE DRAWINGS

FIG. 1 is a graphical representation showing that viral capsid primed CD4⁺ T cells produce IL-10 and are necessary for E7 specific IFN-γ inhibition. In panel (A), groups of 3-5 C57BL/6J mice were immunized with L1 VLPs on day 0 and 14 with or without alum, or were left unimmunized. Lymph nodes lymphocytes were collected on day 21 and exposed to 10 μg/mL L1 VLPs for 48 hours. Supernatants were assayed by ELISA for IL-10. In panel (B), animals were immunized twice with L1 VLPs with alum, and lymph node and spleen lymphocytes exposed to VLPs as for (A), with or without addition of 15 μg/mL of anti-CD4 (GK1.5), anti-CD8 (2.43), or an isotype control antibody. Supernatants were analyzed for IL-10 by ELISA. Results are mean ±SEM of individual mice for splenocytes and of triplicate assays of pooled local lymph node cells. In panel (C), animals were immunized twice with L1 VLPs as in (A), and 500 μL of 1 mg/mL anti-CD4 (GK1.5) or normal rat serum (NRS) given i.p. consecutively for 3 days from day 21. Depletion and recovery of CD4⁺ T cells was monitored by FACS analysis of blood at day 25 and day 43. In panel (D), animals depleted (αCD4) or mock depleted (NRS) of CD4⁺ cells as in (C) were immunized with L1 E7 VLPs on day 44 and 58 (L1/L1E7). Previously untreated animals were similarly immunized with L1E7 VLPs (L1E7). Induction of E7 specific IFN-γ secreting CD8⁺ T cells in spleen and lymph nodes was assessed by ELISPOT on day 55.

FIG. 2 is a graphical representation showing that CD4⁺ T cells from VLP and alum immunized mice suppress E7 specific IFN-γ secretion in vitro. In panel (A), 10⁵ CD11c⁺ dendritic cells from C57BL/6J mice were exposed to 40 μg/mL of BPV1 L1E7 VLPs, BPV1 L1VLPs or HPV6 L1VLPs respectively for 18 hours. After extensive washing, 5×10⁵ E7 TCR transgenic T cells were added and cultured for 48 hours. Supernatants were measured for IFN-γ by ELISA. ³H thymidine was added and T cell proliferation assessed as ³H incorporation. In panel (B), CD11c+ cells (10⁵) were exposed to BPV1 L1E7 VLPs for 18 hours, or cultured without antigen. After washing, CD4⁺ T cells (10⁵) from BPV1 L1 VLP immunized mice, or from unimmunized mice, were added as shown. 5×10⁵ E7 TCR cells, and GK1.5 anti-CD4 antibody at a final concentration of 15 μg/mL were added, and T cell proliferation and IFN-γ secretion were assayed as above. In panel (C), CD11c+ cells (10⁵) were exposed to BPV1 L1E7 VLPs or BPV1 L1 VLPs for 18 hours. After washing, CD4⁺ T cells (10⁵) from BPV1 L1 VLP with Alum immunized or from OVT with Alum, a MHC II restricted peptide of OVA immunized mice, or from unimmunized mice, were added as shown. 5×10⁵ E7 TCR cells were added, and T cell proliferation and IFN-γ secretion were assayed as above. Results are mean ±SEM from triplicate samples. In panel (D), CD11c⁺ cells (10⁵) were exposed to either BPV1 L1E7 VLPs, or BPV1 L1 VLPs for 18 hours. After washing, CD4⁺ T cells (10⁵) from BPV1 L1 VLP with Alum immunized mice, or from OVT with Alum immunized mice, were added as shown. Supernatants were collected and IL-10 secretion was measured by ELISA. Results are means ±SEM from triplicate samples.

FIG. 3 is a graphical representation showing that neutralization of IL-10 restores E7 specific IFN-γ secretion. Panel (A) shows in vitro neutralization. CD 11c⁺ cells (10⁵) from C57BL/6J mice were exposed to 40 μg/mL of E7VLPs for 18 hours. CD4 cells (10⁵) from mice immunized with L1VLP and Alum, and anti-IL-10 antibodies as shown, were added for 18 hours. E7 TCR transgenic T cells (5×10⁵) were then added for 48 hours. Supernatants were collected for cytokine ELISA, and T cell proliferation was assessed as ³H thymidine incorporation. Panel (B) shows in vivo neutralization. Groups of 3 C57BL/6J mice were immunized with L1 VLPs on day 0 and 14. Mice were given either 0.5 mg of anti-IL-10R or normal rat serum i.p. as shown on day 20 and 21, and immunized with E7 VLPs on day 21. On day 34 and 35, IL-10R antibody administration and VLP immunization was repeated. On day 41, spleen and local draining lymph nodes were collected and E7 peptide specific IFN-γ ELISPOT performed.

FIG. 4 is a graphical representation showing that dendritic cells (DCs) exposed to VLP primed CD4⁺ T cells instruct CD8⁺ T cells to secrete IL-5 in response to antigen. In panel (A), CD11c⁺ cells from C57BL/6J mice (10⁵) were exposed to 40 μg/mL of either L1E7VLPs or L1VLPs for 18 hours. They were then left untreated or exposed for 18 hours to CD4⁺ cells (10⁵) from mice immunized with OVT with Alum or L1VLP with Alum as shown. E7 TCR transgenic T cells (5×10⁵) were added for 48 hours. Culture supernatants were collected for IL-5 ELISA. In panel (B), CD11c⁺ cells were incubated with 40 μg/mL of L1E7 VLPs, then either left untreated (a,b) or exposed for 18 hours to CD4⁺ cells from L1VLP (c,d) or L1VLP and Alum (e,f) immunized C57BL/6J mice. CD4⁺ cells were then depleted by positive selection (FACS profile—AFTER) and 5×10⁵ E7 specific or unrelated T cells added for 48 hours; T cell proliferation (a,c,e) was assayed and IL-5 level (b,d,f) were measured by ELISA

FIG. 5 is a graphical representation showing that CpG stimulation cannot overcome suppression of E7 specific IFN-γ secretion by IL-10 secreting CD4 cells Group of 3 C57BL/6J mice were immunized with L1 VLPs on day 0 and 14 (L1/L1E7) or were left unimmunized (L1E7). On day 21 and 35, all groups were immunized with E7 VLPs; one group was also given 10 μg/mL of CpG as shown. 6 days after final immunization, spleen and draining lymph nodes were collected, and E7 specific IFN-γ ELISPOT assays performed. Results are the mean and 1 S.E.M from 3 mice in each group.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

The term “about” is used herein to refer to conditions (e.g., amounts, concentrations, time etc) that vary by as much as 30%, preferably by as much as 20%, and more preferably by as much as 10%, 9%, 8%, 7%, 6%, 5%, 4%, 3%, 2% or 1% to a specified condition.

By “antigen” is meant all, or part of, a protein, peptide, or other molecule or macromolecule capable of eliciting an immune response in a vertebrate animal, especially a mammal. Such antigens are also reactive with antibodies from animals immunized with that protein, peptide, or other molecule or macromolecule.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity.

By “autologous” is meant something (e.g., cells, tissues etc) derived from the same organism.

The term “allogeneic” as used herein refers to cells, tissues, organisms etc that are of different genetic constitution.

By “biologically active fragment” is meant a fragment of a full-length parent polypeptide which fragment retains an activity of the parent polypeptide. As used herein, the term “biologically active fragment” includes deletion mutants and small peptides, for example of at least 6, 7, 8, 9, 10, 11, 12, 13, 14, 15, 16, 17, 18, 19, 20, 21, 22, 23, 24, 25, 26, 27, 28, 29 or 30 contiguous amino acids, which comprise an activity of the parent polypeptide. Peptides of this type may be obtained through the application of standard recombinant nucleic acid techniques or synthesized using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of a polypeptide of the invention with proteinases such as endoLys-C, endoArg-C, endoGlu-C and staphylococcus V8-protease. The digested fragments can be purified by, for example, high performance liquid chromatographic (HPLC) techniques.

As used herein, a “cellular composition,” “cellular vaccine” or “cellular immunogen” refers to a composition comprising at least one cell population as an active ingredient.

As used herein, the term “cis-acting sequence” or “cis-regulatory region” or similar term shall be taken to mean any sequence of nucleotides which is derived from an expressible genetic sequence wherein the expression of the genetic sequence is regulated, at least in part, by the sequence of nucleotides. Those skilled in the art will be aware that a cis-regulatory region may be capable of activating, silencing, enhancing, repressing or otherwise altering the level of expression and/or cell-type-specificity and/or developmental specificity of any structural gene sequence.

Throughout this specification, unless the context requires otherwise, the words “comprise,” “comprises” and “comprising” will be understood to imply the inclusion of a stated step or element or group of steps or elements but not the exclusion of any other step or element or group of steps or elements.

By “corresponds to” or “corresponding to” is meant an antigen which encodes an amino acid sequence that displays substantial similarity to an amino acid sequence in a target antigen. In general the antigen will display at least about 30, 40, 50, 55, 60, 65, 70, 75, 80, 85, 90, 91, 92, 93, 94, 95, 96, 97, 98, 99% similarity to at least a portion of the target antigen.

As used herein, “culturing,” “culture” and the like refer to the set of procedures used in vitro where a population of cells (or a single cell) is incubated under conditions which have been shown to support the growth or maintenance of the cells in vitro. The art recognizes a wide number of formats, media, temperature ranges, gas concentrations etc. which need to be defined in a culture system. The parameters will vary based on the format selected and the specific needs of the individual who practices the methods herein disclosed. However, it is recognized that the determination of culture parameters is routine in nature.

By “effective amount,” in the context of modulating an immune response or treating or preventing a disease or condition, is meant the administration of that amount of composition to an individual in need thereof, either in a single dose or as part of a series, that is effective for achieving that modulation, treatment or prevention. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

By “expression vector” is meant any autonomous genetic element capable of directing the synthesis of a protein encoded by the vector. Such expression vectors are known by practitioners in the art.

The term “gene” is used in its broadest context to include both a genomic DNA region corresponding to the gene as well as a cDNA sequence corresponding to exons or a recombinant molecule engineered to encode a functional form of a product.

By “derivative” is meant a polypeptide that has been derived from the basic sequence by modification, for example by conjugation or complexing with other chemical moieties or by post-translational modification techniques as would be understood in the art. The term “derivative” also includes within its scope alterations that have been made to a parent sequence including additions, or deletions that provide for functionally equivalent molecules.

To enhance immune response (“immunoenhancement”), as is well-known in the art, means to increase the animal's capacity to respond to foreign or disease-specific antigens (e.g., cancer antigens) i.e., those cells primed to attack such antigens are increased in number, activity, and ability to detect and destroy the those antigens. Strength of immune response is measured by standard tests including: direct measurement of peripheral blood lymphocytes by means known to the art; natural killer cell cytotoxicity assays (see, e.g., Provinciali M. et al (1992, J. Immunol. Meth. 155: 19-24), cell proliferation assays (see, e.g., Vollenweider, I. And Groseurth, P. J. (1992, J. Immunol. Meth. 149: 133-135), immunoassays of immune cells and subsets (see, e.g., Loeffler, D. A., et al. (1992, Cytom. 13: 169-174); Rivoltini, L., et al. (1992, Can. Immunol. Immunother. 34: 241-251); or skin tests for cell-mediated immunity (see, e.g., Chang, A. E. et al (1993, Cancer Res. 53: 1043-1050). Any statistically significant increase in strength of immune response as measured by the foregoing tests is considered “enhanced immune response” “immunoenhancement” or “immunopotentiation” as used herein. Enhanced immune response is also indicated by physical manifestations such as fever and inflammation, as well as healing of systemic and local infections, and reduction of symptoms in disease, i.e., decrease in tumor size, alleviation of symptoms of a disease or condition including, but not restricted to, leprosy, tuberculosis, malaria, naphthous ulcers, herpetic and papillomatous warts, gingivitis, arthrosclerosis, the concomitants of AIDS such as Kaposi's sarcoma, bronchial infections, and the like. Such physical manifestations also define “enhanced immune response” “immunoenhancement” or “immunopotentiation” as used herein.

Reference herein to “immunodeficient” includes reference to any condition in which there is a deficiency in the production of humoral and/or cell-mediated immunity.

Reference herein to “immuno-interactive” includes reference to any interaction, reaction, or other form of association between molecules and in particular where one of the molecules is, or mimics, a component of the immune system.

“Inactivation” of a cell is used herein to indicate that the cell has been rendered incapable of cell division to form progeny. The cell may nonetheless be capable of response to stimulus, or biosynthesis and/or secretion of cell products such as cytokines. Methods of inactivation are known in the art. Preferred methods of inactivation are treatment with toxins such as mitomycin C, or irradiation. Cells that have been fixed or permeabilized and are incapable of division are also examples of inactivated cells.

By “isolated” is meant material that is substantially or essentially free from components that normally accompany it in its native state.

A composition is “immunogenic” if it is capable of either: a) generating an immune response against an antigen (e.g., a tumor antigen) in a naïve individual; or b) reconstituting, boosting, or maintaining an immune response in an individual beyond what would occur if the compound or composition was not administered. A composition is immunogenic if it is capable of attaining either of these criteria when administered in single or multiple doses.

By “modulating” is meant increasing or decreasing, either directly or indirectly, the level and/or functional activity of a target molecule. For example, an agent may indirectly modulate the said level/activity by interacting with a molecule other than the target molecule. In this regard, indirect modulation of a gene encoding a target polypeptide includes within its scope modulation of the expression of a first nucleic acid molecule, wherein an expression product of the first nucleic acid molecule modulates the expression of a nucleic acid molecule encoding the target polypeptide. In certain embodiments, “modulation” or “modulating” means that a desired/selected response is more efficient (e.g., at least 10%, 20%, 30%, 40%, 50%, 60% or more), more rapid (e.g., at least 10%, 20%, 30%, 40%, 50%, 60% or more), greater in magnitude (e.g., at least 10%, 20%, 30%, 40%, 50%, 60% or more), and/or more easily induced (e.g., at least 10%, 20%, 30%, 40%, 50%, 60% or more) than if the antigen had been used alone.

The term “5′ non-coding region” is used herein in its broadest context to include all nucleotide sequences which are derived from the upstream region of an expressible gene, other than those sequences which encode amino acid residues which comprise the polypeptide product of said gene, wherein 5′ non-coding region confers or activates or otherwise facilitates, at least in part, expression of the gene.

By “obtained from” is meant that a sample such as, for example, a nucleic acid extract or polypeptide extract is isolated from, or derived from, a particular source of the host. For example, the extract may be obtained from a tissue or a biological fluid isolated directly from the host.

The term “oligonucleotide” as used herein refers to a polymer composed of a multiplicity of nucleotide units (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotides and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule may vary depending on the particular application. An oligonucleotide is typically rather short in length, generally from about 10 to 30 nucleotides, but the term can refer to molecules of any length, although the term “polynucleotide” or “nucleic acid” is typically used for large oligonucleotides.

The term “operably connected” or “operably linked” as used herein means placing a structural gene under the regulatory control of a promoter, which then controls the transcription and optionally the translation of the gene. In the construction of heterologous promoter/structural gene combinations, it is generally preferred to position the genetic sequence or promoter at a distance from the gene transcription start site that is approximately the same as the distance between that genetic sequence or promoter and the gene it controls in its natural setting; i.e., the gene from which the genetic sequence or promoter is derived. As is known in the art, some variation in this distance can be accommodated without loss of function. Similarly, the preferred positioning of a regulatory sequence element with respect to a heterologous gene to be placed under its control is defined by the positioning of the element in its natural setting; i.e., the genes from which it is derived.

The terms “patient,” “subject,” “host” or “individual” used interchangeably herein, refer to any subject, particularly a vertebrate subject, and even more particularly a mammalian subject, for whom therapy or prophylaxis is desired. Suitable vertebrate animals that fall within the scope of the present invention include, but are not restricted to, any member of the subphylum Chordata including primates, rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc), marine mammals (e.g., dolphins, whales), reptiles (snakes, frogs, lizards etc), and fish. A preferred subject is a human in need of treatment or prophylaxis for a condition or disease, which is associated with the presence or aberrant expression of an antigen of interest. However, it will be understood that the aforementioned terms do not imply that symptoms are present.

By “pharmaceutically-acceptable carrier” is meant a solid or liquid filler, diluent or encapsulating substance that may be safely used in topical or systemic administration.

The term “pharmaceutically compatible salt” as used herein refers to a salt which is toxicologically safe for human and animal administration. This salt may be selected from a group including hydrochlorides, hydrobromides, hydroiodides, sulfates, bisulfates, nitrates, citrates, tartrates, bitartrates, phosphates, malates, maleates, napsylates, fumarates, succinates, acetates, terephthalates, pamoates and pectinates.

The term “polynucleotide” or “nucleic acid” as used herein designates mRNA, RNA, cRNA, cDNA or DNA. The term typically refers to oligonucleotides greater than 30 nucleotides in length.

The terms “polynucleotide variant” and “variant” refer to polynucleotides displaying substantial sequence identity with a reference polynucleotide sequence or polynucleotides that hybridize with a reference sequence under stringent conditions that are defined herein. These terms also encompasses polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains the biological function or activity of the reference polynucleotide. The terms “polynucleotide variant” and “variant” also include naturally occurring allelic variants.

“Polypeptide,” “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers.

The term “polypeptide variant” refers to polypeptides which vary from a reference polypeptide by the addition, deletion or substitution of at least one amino acid. It is well understood in the art that some amino acids may be changed to others with broadly similar properties without changing the nature of the activity of the polypeptide. Accordingly, polypeptide variants as used herein encompass polypeptides that have similar activities to a parent polypeptide selected from an IFN α, an IFN β, an IFN γ, a B7-1 molecule and a B7-2 molecule. Preferred variant polypeptides comprise conservative amino acid substitutions. Exemplary conservative substitutions in a polypeptide may be made according to the following table:

TABLE A

| ORIGINAL RESIDUE | EXEMPLARY SUBSTITUTIONS |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile, |
| Phe | Met, Leu, Tyr |
| Ser | Thr |
| Thr | Ser |
| Trp | Tyr |
| Tyr | Trp, Phe |
| Val | Ile, Leu |

Substantial changes in function are made by selecting substitutions that are less conservative than those shown in TABLE A. Other replacements would be non-conservative substitutions and relatively fewer of these may be tolerated. Generally, the substitutions which are likely to produce the greatest changes in a polypeptide's properties are those in which (a) a hydrophilic residue (e.g., Ser or Asn) is substituted for, or by, a hydrophobic residue (e.g., Ala, Leu, Ile, Phe or Val); (b) a cysteine or proline is substituted for, or by, any other residue; (c) a residue having an electropositive side chain (e.g., Arg, His or Lys) is substituted for, or by, an electronegative residue (e.g., Glu or Asp) or (d) a residue having a smaller side chain (e.g., Ala, Ser) or no side chain (e.g., Gly) is substituted for, or by, one having a bulky side chain (e.g., Phe or Trp).

Reference herein to a “promoter” is to be taken in its broadest context and includes the transcriptional regulatory sequences of a classical genomic gene, including the TATA box which is required for accurate transcription initiation, with or without a CCAAT box sequence and additional regulatory elements (i.e., upstream activating sequences, enhancers and silencers) which alter gene expression in response to developmental and/or environmental stimuli, or in a tissue-specific or cell-type-specific manner. A promoter is usually, but not necessarily, positioned upstream or 5′, of a structural gene, the expression of which it regulates. Furthermore, the regulatory elements comprising a promoter are usually positioned within 2 kb of the start site of transcription of the gene. Preferred promoters according to the invention may contain additional copies of one or more specific regulatory elements to further enhance expression in a cell, and/or to alter the timing of expression of a structural gene to which it is operably connected.

The term “recombinant polynucleotide” as used herein refers to a polynucleotide formed in vitro by the manipulation of nucleic acid into a form not normally found in nature. For example, the recombinant polynucleotide may be in the form of an expression vector. Generally, such expression vectors include transcriptional and translational regulatory nucleic acid operably linked to the nucleotide sequence.

By “recombinant polypeptide” is meant a polypeptide made using recombinant techniques, i.e., through the expression of a recombinant polynucleotide.

As used herein “stimulating” an immune or immunological response refers to the administration of a composition that initiates, boosts, or maintains the capacity for the host's immune system to react to a target antigen, such as a foreign molecule, an allogeneic cell, or a tumor cell, at a level higher than would otherwise occur in the absence of the composition. Stimulating a “primary” immune response refers herein to eliciting specific immune reactivity in a subject in which previous reactivity was not detected; for example, due to lack of exposure to the target antigen, refractoriness to the target, or immune suppression. Stimulating a “secondary” response refers to the reinitiation, boosting, or maintenance of reactivity in a subject in which previous reactivity was detected; for example, due to natural immunity, spontaneous immunization, or treatment using one or several compositions or procedures.

By “treatment,” “treat,” “treated” and the like is meant to include both therapeutic and prophylactic treatment.

By “vector” is meant a nucleic acid molecule, preferably a DNA molecule derived, for example, from a plasmid, bacteriophage, or plant virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art.

### 2. Compositions

The present invention stems at least in part from the determination that a subject's secondary immune response to a target antigen can be compromised by the generation of antigen specific IL-10 secreting CD4⁺ T cells, which are produced during a primary response to the target antigen, and which prevent naïve antigen specific CD8⁺ cells acquiring a mature phenotype including cytotoxic function and the ability to secrete IFN-γ. The present inventors have also determined that temporary neutralization of IL-10 either in vitro or in vivo restored the capacity of the subject to mount a CD8⁺ IFN-γ response to the target antigen following appropriate immunization. Based on these observations, the present inventors propose that more efficacious immune responses, whether primary or secondary, which maintain a subject's capacity to raise or maintain an immune response against a target antigen, can be achieved using compositions that comprise an inhibitor of IL-10 function and an immune stimulator that stimulates or otherwise enhances an immune response to the target antigen.

2.1 Inhibitors of IL-10 Function

The inhibitor of IL-10 function includes any molecule or compound that directly or indirectly binds or physically associates with IL-10 or its receptor(s) and that suitably blocks, inhibits or otherwise antagonizes at least one of its functions or activities (e.g., binding to or interaction with one or more surface molecules (e.g., receptors) present on white blood cells, especially lymphocytes and more especially T lymphocytes). The binding or association may involve the formation of an induced magnetic field or paramagnetic field, covalent bond formation, an ionic interaction such as, for example, occur in an ionic lattice, a hydrogen bond or alternatively, a van der Waals interaction such as, for example, a dipole-dipole interaction, dipole-induced-dipole interaction, induced-dipole-induced-dipole interaction or a repulsive interaction or any combination of the above forces of attraction. In certain embodiments, the inhibitor of IL-10 function is an antigen-binding molecule that is immuno-interactive with at least a portion of IL-10. In these embodiments, the antigen-binding molecules can be immuno-interactive with an active or an inactive form of IL-10, the difference being that antigen-binding molecules to the active cytokine are more likely to recognize epitopes that are only present in the active conformation. Illustrative antigen-binding molecules and methods for their production are described in U.S. Pat. Nos. 5,837,232; 5,837,293; and 6,239,260

In other embodiments, the inhibitor of IL-10 function is any molecule capable of specifically preventing activation of cellular receptors for IL-10. For example, such an inhibitor can be an antigen-binding molecule that is immuno-interactive with an IL-10 receptor, representative examples of which are described in U.S. Pat. No. 5,863,796. Alternatively, inhibitors of this type can be selected from soluble or defective IL-10 receptors or soluble IL-10 receptor subunits. Representative receptors of this type are described, for example, in U.S. Pat. Nos. 5,843,697 and 6,423,500, U.S Patent Application Publication Nos. 20040204351 and 20050064464, Tan et al. (1995, J. Biol. Chem. 270: 12906) and in GenBank Accession Nos. U00672 or NM_{—}001558. In other embodiments, the inhibitor of IL-10 function is an IL-10 receptor that is expressed on the surface of a cell (e.g., a bacterial cell) as described, for example, in U.S Patent Application Publication No. 20020019043.

In some embodiments, the inhibitor of IL-10 function is a nucleic acid that inhibits the expression of an IL-10 gene or the functional activity of its expression products. In illustrative examples of this type, the inhibitor reduces or abrogates IL-10 gene expression and includes, but is not restricted to, oligoribonucleotide sequences, including anti-sense RNA and DNA molecules and ribozymes, that function to inhibit the translation of a messenger RNA that stimulates the IL-10 signaling pathway. Anti-sense RNA and DNA molecules act to directly block the translation of mRNA by binding to targeted mRNA and preventing protein translation. In regard to antisense DNA, oligodeoxyribonucleotides derived from the translation initiation site, e.g., between −10 and +10 regions are preferred. Illustrative antisense oligonucleotides with specificity to the IL-10 gene and its transcripts are described in U.S. Pat. No. 6,184,372. Alternatively, RNA molecules that mediate RNA interference (RNAi) of a target gene or gene transcript can be used to reduce or abrogate gene expression. RNAi refers to interference with or destruction of the product of a target gene by introducing a single stranded, and typically a double stranded RNA (dsRNA) that is homologous to the transcript of a target gene. Thus, in some embodiments, dsRNA per se and especially dsRNA-producing constructs corresponding to at least a portion of an IL-10 gene may be used to reduce or abrogate its expression. RNAi-mediated inhibition of gene expression may be accomplished using any of the techniques reported in the art, for instance by transfecting a nucleic acid construct encoding a stem-loop or hairpin RNA structure into the genome of the target cell, or by expressing a transfected nucleic acid construct having homology for a target gene from between convergent promoters, or as a head to head or tail to tail duplication from behind a single promoter. Any similar construct may be used so long as it produces a single RNA having the ability to fold back on itself and produce a dsRNA, or so long as it produces two separate RNA transcripts which then anneal to form a dsRNA having homology to a target gene. Alternatively, RNA molecules of about 21 to about 23 nucleotides, which direct cleavage of specific mRNA to which they correspond, as for example described by Tuschl et al. in U.S. Patent Application No. 20020086356, can be utilized for mediating RNAi. Such 21-23 nt RNA molecules can comprise a 3′ hydroxyl group, can be single-stranded or double stranded (as two 21-23 nt RNAs) wherein the dsRNA molecules can be blunt ended or comprise overhanging ends (e.g., 5′, 3′).

In other embodiments, the inhibitor of IL-10 function is a small molecule modulator of IL-10, illustrative examples of which include beta-alethine, beta-alanyl taurine, carbobenzoxy beta-alanyl taurine, and various other aminothiols and amino phosphates that fall within the compounds disclosed in U.S. Pat. No. 6,451,853 as well as substituted isoquinolines, isochromanones and isothiochromanones as described, for example, in U.S. Pat. No. 6,723,736.

Desirably, the inhibitor of IL-10 function is non-toxic to the host with minimal or negligible side effects.

2.2 Immune-Modulating Agents

2.2.1 Antigens

The present invention contemplates the use in the compositions of the invention of any antigen that corresponds to at least a portion of a target antigen of interest for stimulating an immune response to the target antigen. Such an antigen may be in soluble form (e.g., peptide, polypeptide or a nucleic acid molecule from which a peptide or polypeptide is expressible) or in the form of whole cells or attenuated pathogen preparations (e.g., attenuated virus or bacteria) or it may be presented by antigen-presenting cells as described in more detail below.

Target antigens useful in the present invention are typically proteinaceous molecules, representative examples of which include polypeptides and peptides. Such molecules may also include, for example, a non-proteinaceous moiety such as but not limited to simple intermediary metabolites, sugars, lipids, and hormones as well as macromolecules such as complex carbohydrates, phospholipids and nucleic acids. Target antigens may be selected from endogenous antigens produced by a host or exogenous antigens that are foreign to the host. Suitable endogenous antigens include, but are not restricted to, cancer or tumor antigens. Non-limiting examples of cancer or tumor antigens include antigens from a cancer or tumor selected from ABLI proto-oncogene, AIDS related cancers, acoustic neuroma, acute lymphocytic leukemia, acute myeloid leukemia, adenocystic carcinoma, adrenocortical cancer, agnogenic myeloid metaplasia, alopecia, alveolar soft-part sarcoma, anal cancer, angiosarcoma, aplastic anemia, astrocytoma, ataxia-telangiectasia, basal cell carcinoma (skin), bladder cancer, bone cancers, bowel cancer, brain stem glioma, brain and CNS tumors, breast cancer, CNS tumors, carcinoid tumors, cervical cancer, childhood brain tumors, childhood cancer, childhood leukemia, childhood soft tissue sarcoma, chondrosarcoma, choriocarcinoma, chronic lymphocytic leukemia, chronic myeloid leukemia, colorectal cancers, cutaneous T-cell lymphoma, dermatofibrosarcoma protuberans, desmoplastic small round cell tumor, ductal carcinoma, endocrine cancers, endometrial cancer, ependymoma, oesophageal cancer, Ewing's Sarcoma, Extra-Hepatic Bile Duct Cancer, Eye Cancer, Eye: Melanoma, Retinoblastoma, Fallopian Tube cancer, Fanconi anemia, fibrosarcoma, gall bladder cancer, gastric cancer, gastrointestinal cancers, gastrointestinal-carcinoid-tumor, genitourinary cancers, germ cell tumors, gestational-trophoblastic-disease, glioma, gynecological cancers, haematological malignancies, hairy cell leukemia, head and neck cancer, hepatocellular cancer, hereditary breast cancer, histiocytosis, Hodgkin's disease, human papillomavirus, hydatidiform mole, hypercalcemia, hypopharynx cancer, intraocular melanoma, islet cell cancer, Kaposi's sarcoma, kidney cancer, Langerhan's cell histiocytosis, laryngeal cancer, leiomyosarcoma, leukemia, Li-Fraumeni syndrome, lip cancer, liposarcoma, liver cancer, lung cancer, lymphedema, lymphoma, Hodgkin's lymphoma, non-Hodgkin's lymphoma, male breast cancer, malignant-rhabdoid tumor of kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloproliferative disorders, nasal cancer, nasopharyngeal cancer, nephroblastoma, neuroblastoma, neurofibromatosis, Nijmegen breakage syndrome, non-melanoma skin cancer, non-small-cell-lung-cancer (NSCLC), ocular cancers, esophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal tumours, pituitary cancer, polycythemia vera, prostate cancer, rare cancers and associated disorders, renal cell carcinoma, retinoblastoma, rhabdomyosarcoma, Rothmund-Thomson syndrome, salivary gland cancer, sarcoma, schwannoma, Sezary syndrome, skin cancer, small cell lung cancer (SCLC), small intestine cancer, soft tissue sarcoma, spinal cord tumors, squamous-cell-carcinoma-(skin), stomach cancer, synovial sarcoma, testicular cancer, thymus cancer, thyroid cancer, transitional-cell-cancer-(bladder), transitional-cell-cancer-(renal-pelvis−/−ureter), trophoblastic cancer, urethral cancer, urinary system cancer, uroplakins, uterine sarcoma, uterus cancer, vaginal cancer, vulva cancer, Waldenstrom's macroglobulinemia, Wilms' tumor. In certain embodiments, the cancer or tumor relates to melanoma. Illustrative examples of melanoma-related antigens include melanocyte differentiation antigen (e.g., gp100, MART, Melan-A/MART-1, TRP-1, Tyros, TRP2, MCIR, MUC1F, MUC1R or a combination thereof) and melanoma-specific antigens (e.g., BAGE, GAGE-1, gp100In4, MAGE-1 (e.g., GenBank Accession No. X54156 and AA494311), MAGE-3, MAGE4, PRAME, TRP2IN2, NYNSO1a, NYNSO1b, LAGE1, p97 melanoma antigen (e.g., GenBank Accession No. M12154) p5 protein, gp75, oncofetal antigen, GM2 and GD2 gangliosides, cdc27, p21ras, gp100^{Pmel117} or a combination thereof. Other tumour-specific antigens include, but are not limited to: etv6, aml1, cyclophilin b (acute lymphoblastic leukemia); Ig-idiotype (B cell lymphoma); E-cadherin, α-catenin, β-catenin, γ-catenin, p120ctn (glioma); p21ras (bladder cancer); p21ras (biliary cancer); MUC family, HER2/neu, c-erbB-2 (breast cancer); p53, p21ras (cervical carcinoma); p21ras, HER2/neu, c-erbB-2, MUC family, Cripto-1protein, Pim-1 protein (colon carcinoma); Colorectal associated antigen (CRC)-CO17-1A/GA733, APC (colorectal cancer); carcinoembryonic antigen (CEA) (colorectal cancer; choriocarcinoma); cyclophilin b (epithelial cell cancer); HER2/neu, c-erbB-2, ga733 glycoprotein (gastric cancer); α-fetoprotein (hepatocellular cancer); Imp-1, EBNA-1 (Hodgkin's lymphoma); CEA, MAGE-3, NY-ESO-1 (lung cancer); cyclophilin b (lymphoid cell-derived leukemia); MUC family, p21ras (myeloma); HE/neu, c-erbB-2 (non-small cell lung carcinoma); Imp-1, EBNA-1 (nasopharyngeal cancer); MUC family, HER2/neu, c-erbB-2, MAGE-A4, NY-ESO-1 (ovarian cancer); Prostate Specific Antigen (PSA) and its antigenic epitopes PSA-1, PSA-2, and PSA-3, PSMA, HER2/neu, c-erbB-2, ga733 glycoprotein (prostate cancer); HER2/neu, c-erbB-2 (renal cancer); viral products such as human papillomavirus proteins (squamous cell cancers of the cervix and esophagus); NY-ESO-1 (testicular cancer); and HTLV-1 epitopes (T cell leukemia).

Foreign or exogenous antigens are suitably selected from antigens of pathogenic organisms. Exemplary pathogenic organisms include, but are not limited to, viruses, bacteria, fungi parasites, algae and protozoa and amoebae. Illustrative viruses include viruses responsible for diseases including, but not limited to, measles, mumps, rubella, poliomyelitis, hepatitis A, B (e.g., GenBank Accession No. E02707), and C (e.g., GenBank Accession No. E06890), as well as other hepatitis viruses, influenza, adenovirus (e.g., types 4 and 7), rabies (e.g., GenBank Accession No. M34678), yellow fever, Epstein-Barr virus and other herpesviruses such as papillomavirus, Ebola virus, influenza virus, Japanese encephalitis (e.g., GenBank Accession No. E07883), dengue (e.g., GenBank Accession No. M24444), hantavirus, Sendai virus, respiratory syncytial virus, othromyxoviruses, vesicular stomatitis virus, visna virus, cytomegalovirus and human immunodeficiency virus (HIV) (e.g., GenBank Accession No. U18552). Any suitable antigen derived from such viruses are useful in the practice of the present invention. For example, illustrative retroviral antigens derived from HIV include, but are not limited to, antigens such as gene products of the gag, pol, and env genes, the Nef protein, reverse transcriptase, and other HIV components. Illustrative examples of hepatitis viral antigens include, but are not limited to, antigens such as the S, M, and L proteins of hepatitis B virus, the pre-S antigen of hepatitis B virus, and other hepatitis, e.g., hepatitis A, B, and C, viral components such as hepatitis C viral RNA. Illustrative examples of influenza viral antigens include; but are not limited to, antigens such as hemagglutinin and neurarninidase and other influenza viral components. Illustrative examples of measles viral antigens include, but are not limited to, antigens such as the measles virus fusion protein and other measles virus components. Illustrative examples of rubella viral antigens include, but are not limited to, antigens such as proteins E1 and E2 and other rubella virus components; rotaviral antigens such as VP7sc and other rotaviral components. Illustrative examples of cytomegaloviral antigens include, but are not limited to, antigens such as envelope glycoprotein B and other cytomegaloviral antigen components. Non-limiting examples of respiratory syncytial viral antigens include antigens such as the RSV fusion protein, the M2 protein and other respiratory syncytial viral antigen components. Illustrative examples of herpes simplex viral antigens include, but are not limited to, antigens such as immediate early proteins, glycoprotein D, and other herpes simplex viral antigen components. Non-limiting examples of varicella zoster viral antigens include antigens such as 9PI, gpII, and other varicella zoster viral antigen components. Non-limiting examples of Japanese encephalitis viral antigens include antigens such as proteins E, M-E, M-E-NS 1, NS 1, NS 1-NS2A, 80% E, and other Japanese encephalitis viral antigen components. Representative examples of rabies viral antigens include, but are not limited to, antigens such as rabies glycoprotein, rabies nucleoprotein and other rabies viral antigen components. Illustrative examples of papillomavirus antigens include, but are not limited to, the L1 and L2 capsid proteins as well as the E6/E7 antigens associated with cervical cancers, See Fundamental Virology, Second Edition, eds. Fields, B. N. and Knipe, D. M., 1991, Raven Press, New York, for additional examples of viral antigens.

Illustrative examples of fungi include Acremonium spp., Aspergillus spp., Basidiobolus spp., Bipolaris spp., Blastomyces dermatidis, Candida spp., Cladophialophora carrionii, Coccoidiodes immitis, Conidiobolus spp., Cryptococcus spp., Curvularia spp., Epidermophyton spp., Exophiala jeanselinei, Exserohilum spp., Fonsecaea compacta, Fonsecaea pedrosoi, Fusarium oxysporum, Fusarium solani, Geotrichum candidum, Histoplasma capsulatum var. capsulatum, Histoplasma capsulatum var. duboisii, Hortaea werneckii, Lacazia loboi, Lasiodiplodia theobromae, Leptosphaeria senegalensis, Madurella grisea, Madurella niycetomatis, Malasseziafurfur, Microsporum spp., Neotestudina rosatii, Onychocola canadensis, Paracoccidioides brasiliensis, Phialophora verrucosa, Piedraia hortae, Piedra iahortae, Pityriasis versicolor, Pseudallesheria boydii, Pyrenochaeta romeroi, Rhizopus arrhizus, Scopulariopsis brevicaulis, Scytalidium dimidiatum, Sporothrix schenckii, Trichophyton spp., Trichosporon spp., Zygomcete fungi, Absidia corymbifera, Rhizomucor pusillus and Rhizopus arrhizus. Thus, representative fungal antigens that can be used in the compositions and methods of the present invention include, but are not limited to, candida fungal antigen components; histoplasma fungal antigens such as heat shock protein 60 (HSP60) and other histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components.

Illustrative examples of bacteria include bacteria that are responsible for diseases including, but not restricted to, diphtheria (e.g., Corynebacterium diphtheria), pertussis (e.g., Bordetella pertussis, GenBank Accession No. M35274), tetanus (e.g., Clostridium tetani, GenBank Accession No. M64353), tuberculosis (e.g., Mycobacterium tuberculosis), bacterial pneumonias (e.g., Haemophilus influenzae.), cholera (e.g., Vibrio cholerae), anthrax (e.g., Bacillus anthracis), typhoid, plague, shigellosis (e.g., Shigella dysenteriae), botulism (e.g., Clostridium botulinum), salmonellosis (e.g., GenBank Accession No. L03833), peptic ulcers (e.g., Helicobacter pylori), Legionnaire's Disease, Lyme disease (e.g., GenBank Accession No. U59487), Other pathogenic bacteria include Escherichia coli, Clostridium perfringens, Pseudomonas aeruginosa, Staphylococcus aureus and Streptococcus pyogenes. Thus, bacterial antigens which can be used in the compositions and methods of the invention include, but are not limited to: pertussis bacterial antigens such as pertussis toxin, filamentous hemagglutinin, pertactin, F M2, FIM3, adenylate cyclase and other pertussis bacterial antigen components; diphtheria bacterial antigens such as diphtheria toxin or toxoid and other diphtheria bacterial antigen components; tetanus bacterial antigens such as tetanus toxin or toxoid and other tetanus bacterial antigen components, streptococcal bacterial antigens such as M proteins and other streptococcal bacterial antigen components; gram-negative bacilli bacterial antigens such as lipopolysaccharides and other gram-negative bacterial antigen components; Mycobacterium tuberculosis bacterial antigens such as mycolic acid, heat shock protein 65 (HSP65), the 30 kDa major secreted protein, antigen 85A and other mycobacterial antigen components; Helicobacter pylori bacterial antigen components, pneumococcal bacterial antigens such as pneumolysin, pneumococcal capsular polysaccharides and other pnermiococcal bacterial antigen components; Haemophilus influenza bacterial antigens such as capsular polysaccharides and other Haemophilus influenza bacterial antigen components; anthrax bacterial antigens such as anthrax protective antigen and other anthrax bacterial antigen components; rickettsiae bacterial antigens such as rompA and other rickettsiae bacterial antigen component. Also included with the bacterial antigens described herein are any other bacterial, mycobacterial, mycoplasmal, rickettsial, or chlamydial antigens.

Illustrative examples of protozoa include protozoa that are responsible for diseases including, but not limited to, malaria (e.g., GenBank Accession No. X53832), hookworm, onchocerciasis (e.g., GenBank Accession No. M27807), schistosomiasis (e.g., GenBank Accession No. LOS 198), toxoplasmosis, trypanosomiasis, leishmaniasis, giardiasis (GenBank Accession No. M33641), amoebiasis, filariasis (e.g., GenBank Accession No. J03266), borreliosis, and trichinosis. Thus, protozoal antigens which can be used in the compositions and methods of the invention include, but are not limited to: plasmodium falciparum antigens such as merozoite surface antigens, sporozoite surface antigens, circumsporozoite antigens, gametocyte/gamete surface antigens, blood-stage antigen pf 155/RESA and other plasmodial antigen components; toxoplasma antigens such as SAG-1, p30 and other toxoplasmal antigen components; schistosomae antigens such as glutathione-S-transferase, paramyosin, and other schistosomal antigen components; leishmania major and other leishmaniae antigens such as gp63, lipophosphoglycan and its associated protein and other leishmanial antigen components; and trypanosoma cruzi antigens such as the 75-77 kDa antigen, the 56 kDa antigen and other trypanosomal antigen components.

The present invention also contemplates toxin components as antigens, illustrative examples of which include staphylococcal enterotoxins, toxic shock syndrome toxin; retroviral antigens (e.g., antigens derived from HIV), streptococcal antigens, staphylococcal enterotoxin-A (SEA), staphylococcal enterotoxin-B (SEB), staphylococcal enterotoxin₁₋₃ (SE₁₋₃), staphylococcal enterotoxin-D (SED), staphylococcal enterotoxin-E (SEE) as well as toxins derived from mycoplasma, mycobacterium, and herpes viruses.

An antigen corresponding to at least a portion of the target antigen may be isolated from a natural source or may be prepared by recombinant techniques as known in the art. For example, peptide antigens can be eluted from the MHC and other presenting molecules of antigen-presenting cells obtained from a cell population or tissue for which a modified immune response is desired. The eluted peptides can be purified using standard protein purification techniques known in the art (Rawson et al., 2000, Cancer Res 60(16), 4493-4498). If desired, the purified peptides can be sequenced and synthetic versions of the peptides produced using standard protein synthesis techniques as for example described below. Alternatively, crude antigen preparations can be produced by isolating a sample of a cell population or tissue for which a modified immune response is desired, and either lysing the sample or subjecting the sample to conditions that will lead to the formation of apoptotic cells (e.g., irradiation with ultra violet or with γ rays, viral infection, cytokines or by depriving cells of nutrients in the cell culture medium, incubation with hydrogen peroxide, or with drugs such as dexamethasone, ceramide chemotherapeutics and anti-hormonal agents such as Lupron or Tamoxifen). The lysate or the apoptotic cells can then be used as a source of crude antigen for use in soluble form or for contact with antigen-presenting cells as described in more detail below.

When the antigen is known, it may be conveniently prepared in recombinant form using standard protocols as for example described in: Sambrook, et al., MOLECULAR CLONING. A LABORATORY MANUAL (Cold Spring Harbor Press, 1989), in particular Sections 16 and 17; Ausubel et al., CURRENT PROTOCOLS 1N MOLECULAR BIOLOGY (John Wiley & Sons, Inc. 1994-1998), in particular Chapters 10 and 16; and Coligan et al., CURRENT PROTOCOLS IN PROTEIN SCIENCE (John Wiley & Sons, Inc. 1995-1997), in particular Chapters 1, 5 and 6. Typically, an antigen may be prepared by a procedure including the steps of (a) providing an expression vector from which the target antigen or analogue or mimetic thereof is expressible; (b) introducing the vector into a suitable host cell; (c) culturing the host cell to express recombinant polypeptide from the vector; and (d) isolating the recombinant polypeptide.

In general, the expression vector will comprise an antigen-encoding polynucleotide which is operably connected to a regulatory polynucleotide. The antigen-encoding polynucleotide can be constructed from any suitable parent polynucleotide that codes for an antigen that corresponds to the target antigen of interest. The parent polynucleotide is suitably a natural gene or portion thereof. However, it is possible that the parent polynucleotide is not naturally-occurring but has been engineered using recombinant techniques. The regulatory polynucleotide suitably comprises transcriptional and/or translational control sequences, which will generally be appropriate for the host cell used for expression of the antigen-encoding polynucleotide. Typically, the transcriptional and translational regulatory control sequences include, but are not limited to, a promoter sequence, a 5′ non-coding region, a cis-regulatory region such as a functional binding site for transcriptional regulatory protein or translational regulatory protein, an upstream open reading frame, transcriptional start site, translational start site, and/or nucleotide sequence which encodes a leader sequence, termination codon, translational stop site and a 3′ non-translated region. Constitutive or inducible promoters as known in the art are contemplated by the invention. The promoters may be either naturally occurring promoters, or hybrid promoters that combine elements of more than one promoter. Promoter sequences contemplated by the present invention may be native to the host cell to be introduced or may be derived from an alternative source, where the region is functional in the host cell.

The expression vector may also comprise a 3′ non-translated sequence, which usually refers to that portion of a gene comprising a DNA segment that contains a polyadenylation signal and any other regulatory signals capable of effecting mRNA processing or gene expression. The polyadenylation signal is characterized by effecting the addition of polyadenylic acid tracts to the 3′ end of the mRNA precursor. Polyadenylation signals are commonly recognized by the presence of homology to the canonical form 5′ AATAAA-3′ although variations are not uncommon. The 3′ non-translated regulatory DNA sequence typically includes from about 50 to 1,000 nucleotide base pairs and may contain transcriptional and translational termination sequences in addition to a polyadenylation signal and any other regulatory signals capable of effecting mRNA processing or gene expression.

In certain embodiments, the expression vector further contains a selectable marker gene to allow the selection of transformed host cells. Selection genes are well known in the art and will vary with the host cell used.

The expression vector may also include a fusion partner (typically provided by the expression vector) so that the recombinant polypeptide is expressed as a fusion polypeptide with the fusion partner. The main advantage of fusion partners is that they assist identification and/or purification of said fusion polypeptide. Well known examples of fusion partners include, but are not limited to, glutathione-S-transferase (GST), Fc portion of human IgG, maltose binding protein (MBP) and hexahistidine (HIS₆), which are particularly useful for isolation of the fusion polypeptide by affinity chromatography. For the purposes of fusion polypeptide purification by affinity chromatography, relevant matrices for affinity chromatography are glutathione-, amylose-, and nickel- or cobalt-conjugated resins respectively. Many such matrices are available in “kit” form, such as the QIAexpress™ system (Qiagen) useful with (HIS₆) fusion partners and the Pharmacia GST purification system. Preferably, the fusion partners also have protease cleavage sites, such as for Factor Xₐ or Thrombin, which allow the relevant protease to partially digest the fusion polypeptide of the invention and thereby liberate the recombinant polypeptide of the invention therefrom. The liberated polypeptide can then be isolated from the fusion partner by subsequent chromatographic separation. Fusion partners also include within their scope “epitope tags”, which are usually short peptide sequences for which a specific antibody is available. Well known examples of epitope tags for which specific monoclonal antibodies are readily available include c-Myc, influenza virus hemagglutinin and FLAG tags.

The step of introducing the expression vector into the host cell may be achieved by any suitable method including transfection, transduction of viral vectors, including adenoviral, modified lentiviral and other retroviral vectors, and transformation, the choice of which will be dependent on the host cell employed. Such methods are well known to those of skill in the art.

Recombinant polypeptides may be produced by culturing a host cell transformed with the expression vector under conditions appropriate for protein expression, which will vary with the choice of expression vector and the host cell. This is easily ascertained by one skilled in the art through routine experimentation. Suitable host cells for expression may be prokaryotic or eukaryotic. One preferred host cell for expression of a polypeptide according to the invention is a bacterium. The bacterium used may be Escherichia coli. Alternatively, the host cell may be an insect cell such as, for example, SF9 cells that may be utilized with a baculovirus expression system. In some embodiments, the antigen, which is administered with the inhibitor of IL-10 function, is in the form of a construct or vector from which it is expressible.

Alternatively, the antigen can be synthesized using solution synthesis or solid phase synthesis as described, for example, by Atherton and Sheppard (Solid Phase Peptide Synthesis: A Practical Approach, IRL Press at Oxford University Press, Oxford, England, 1989) or by Roberge et al. (1995, Science 269: 202). The amino acids of the synthesized antigens can be non-naturally occurring or naturally occurring amino acid. Examples of unnatural amino acids and derivatives during peptide synthesis include but are not limited to, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, sarcosine, 2-thienyl alanine and/or D-isomers of amino acids. A list of unnatural amino acids contemplated by the present invention is shown in TABLE B.

TABLE B
NON-CONVENTIONAL AMINO ACID

α-aminobutyric acid
α-amino-α-methylbutyrate
aminocyclopropane-carboxylate
aminoisobutyric acid
aminonorbornyl-carboxylate
cyclohexylalanine
cyclopentylalanine
L-N-methylisoleucine
D-alanine
D-arginine
D-aspartic acid
D-cysteine
D-glutamate
D-glutamic acid
D-histidine
D-isoleucine
D-leucine
D-lysine
D-methionine
D-ornithine
D-phenylalanine
D-proline
D-serine
D-threonine
D-tryptophan
D-tyrosine
D-valine
D-α-methylalanine
D-α-methylarginine
D-α-methylasparagine
D-α-methylaspartate
D-α-methylcysteine
D-α-methylglutamine
D-α-methylhistidine
D-α-methylisoleucine
D-α-methylleucine
D-α-methyllysine
D-α-methylmethionine
D-α-methylornithiine
D-α-methylphenylalanine
D-α-methylproline
D-α-methylserine
D-α-methylthreonine
D-α-methyltryptophan
D-α-methyltyrosine
L-α-methylleucine
L-α-methylmethionine
L-α-methylnorvatine
L-α-methylphenylalanine
L-α-methylserine
L-α-methyltryptophan
L-α-methylvaline
N-(N-(2,2-diphenylethyl carbamylmethyl)glycine
1-carboxy-1-(2,2-diphenyl-ethyl amino)cyclopropane
L-N-methylalanine
L-N-methylarginine
L-N-methylasparagine
L-N-methylaspartic acid
L-N-methylcysteine
L-N-methylglutamine
L-N-methylglutamic acid
L-N-methylhistidine
L-N-methylleucine
L-N-methyllysine
L-N-methylmethionine
L-N-methylnorleucine
L-N-methylnorvaline
L-N-methylornithine
L-N-methylphenylalanine
L-N-methylproline
L-N-medlylserine
L-N-methylthreonine
L-N-methyltryptophan
L-N-methyltyrosine
L-N-methylvaline
L-N-methylethylglycine
L-N-methyl-t-butylglycine
L-norleucine
L-norvaline
α-methyl-aminoisobutyrate
α-methyl-γ-aminobutyrate
α-methylcyclohexylalanine
α-methylcylcopentylalanine
α-methyl-α-napthylalanine
α-methylpenicillamine
N-(4-aminobutyl)glycine
N-(2-aminoethyl)glycine
N-(3-aminopropyl)glycine
N-amino-α-methylbutyrate
α-napthylalanine
N-benzylglycine
N-(2-carbamylediyl)glycine
N-(carbamylmethyl)glycine
N-(2-carboxyethyl)glycine
N-(carboxymethyl)glycine
N-cyclobutylglycine
N-cycloheptylglycine
N-cyclohexylglycine
N-cyclodecylglycine
L-α-methyllysine
L-α-methylnorleucine
L-α-methylornithine
L-α-methylproline
L-α-methylthreonine
L-α-methyltyrosine
L-N-methylhomophenylalanine
N-(N-(3,3-diphenylpropyl carbamylmethyl)glycine

The invention also contemplates modifying peptide antigens using ordinary molecular biological techniques so as to alter their resistance to proteolytic degradation or to optimize solubility properties or to render them more suitable as an immunogenic agent.

Peptide antigens may be of any suitable size that can be utilized to stimulate or inhibit an immune response to a target antigen of interest. A number of factors can influence the choice of peptide size. For example, the size of a peptide can be chosen such that it includes, or corresponds to the size of, T cell epitopes and/or B cell epitopes, and their processing requirements. Practitioners in the art will recognize that class I-restricted T cell epitopes are typically between 8 and 10 amino acid residues in length and if placed next to unnatural flanking residues, such epitopes can generally require 2 to 3 natural flanking amino acid residues to ensure that they are efficiently processed and presented. Class II-restricted T cell epitopes usually range between 12 and 25 amino acid residues in length and may not require natural flanking residues for efficient proteolytic processing although it is believed that natural flanking residues may play a role. Another important feature of class II-restricted epitopes is that they generally contain a core of 9-10 amino acid residues in the middle which bind specifically to class II MHC molecules with flanking sequences either side of this core stabilizing binding by associating with conserved structures on either side of class II MHC antigens in a sequence independent manner. Thus the functional region of class II-restricted epitopes is typically less than about 15 amino acid residues long. The size of linear B cell epitopes and the factors effecting their processing, like class II-restricted epitopes, are quite variable although such epitopes are frequently smaller in size than 15 amino acid residues. From the foregoing, it is advantageous, but not essential, that the size of the peptide is at least 6, 7, 8, 9, 10, 11, 12, 13, 14, 15, 20, 25, 30 amino acid residues. Suitably, the size of the peptide is no more than about 500, 200, 100, 80, 60, 50, 40 amino acid residues. In certain advantageous embodiments, the size of the peptide is sufficient for presentation by an antigen-presenting cell of a T cell and/or a B cell epitope contained within the peptide.

Criteria for identifying and selecting effective antigenic peptides (e.g., minimal peptide sequences capable of eliciting an immune response) can be found in the art. For example, Apostolopoulos et al. (2000, Curr. Opin. Mol. Ther. 2:29-36) discusses the strategy for identifying minimal antigenic peptide sequences based on an understanding of the three dimensional structure of an antigen-presenting molecule and its interaction with both an antigenic peptide and T-cell receptor. Shastri (1996, Curr. Opin. Immunol. 8:271-277) discloses how to distinguish rare peptides that serve to activate T cells from the thousands peptides normally bound to MHC molecules.

In some embodiments, the antigen is delivered in the form of a synthetic construct (or expression vector comprising a polynucleotide that encodes the antigen, which is operably linked to a regulatory polynucleotide. The regulatory polynucleotide suitably comprises transcriptional and/or translational control sequences, which will be compatible for expression in the cell or tissue type of interest. Typically, the transcriptional and translational regulatory control sequences include, but are not limited to, a promoter sequence, a 5′ non-coding region, a cis-regulatory region such as a functional binding site for transcriptional regulatory protein or translational regulatory protein, an upstream open reading frame, ribosomal-binding sequences, transcriptional start site, translational start site, and/or nucleotide sequence which encodes a leader sequence, termination codon, translational stop site and a 3′ non-translated region. Constitutive or inducible promoters as known in the art are contemplated by the invention. The promoters may be either naturally occurring promoters, or hybrid promoters that combine elements of more than one promoter. Promoter sequences contemplated by the present invention may be native to the organism of interest or may be derived from an alternative source, where the region is functional in the chosen organism. The choice of promoter will differ depending on the intended host. For example, promoters which could be used for expression in mammalian cells generally include the metallothionein promoter, which can be induced in response to heavy metals such as cadmium, the β-actin promoter as well as viral promoters such as the SV40 large T antigen promoter, human cytomegalovirus (CMV) immediate early (1E) promoter, rous sarcoma virus LTR promoter, adenovirus promoter, or a HPV promoter, particularly the HPV upstream regulatory region (URR) may also be used. All these promoters are well described and readily available in the art. Alternatively, the promoter may be lineage specific and, in this regard, epithelial-specific promoters are particularly desirable such as, but not limited to, promoters of the following genes transglutaminase type 1, involucrin, loricrin, SPR genes and filagrin as well as those of keratin genes (e.g., K10, K14, K5, K1).

The synthetic construct may also comprise a 3′ non-translated sequence. A 3′ non-translated sequence refers to that portion of a gene comprising a DNA segment that contains a polyadenylation signal and any other regulatory signals capable of effecting mRNA processing or gene expression. The polyadenylation signal is characterized by effecting the addition of polyadenylic acid tracts to the 3′ end of the mRNA precursor. Polyadenylation signals are commonly recognized by the presence of homology to the canonical form 5′ AATAAA-3′ although variations are not uncommon. The 3′ non-translated regulatory DNA sequence preferably includes from about 50 to 1,000 nucleotide base pairs and may contain transcriptional and translational termination sequences in addition to a polyadenylation signal and any other regulatory signals capable of effecting mRNA processing or gene expression.

In some embodiments, the synthetic construct further contains a screenable marker gene to permit identification of cells containing the synthetic construct. Screenable genes (e.g., lacZ, gfp, etc) are well known in the art and will be compatible for expression in a particular cell or tissue type.

It will be understood, however, that expression of protein-encoding polynucleotides in heterologous systems is now well known, and the present invention is not directed to or dependent on any particular vector, transcriptional control sequence or technique for its production. Rather, synthetic polynucleotides prepared according to the methods as set forth herein may be introduced into selected cells or tissues or into a precursors or progenitors thereof in any suitable manner in conjunction with any suitable synthetic construct or vector, and the synthetic polynucleotides may be expressed with known promoters in any conventional manner

The synthetic constructs can be introduced into suitable host cells for expression using any of a number of non-viral or viral gene delivery vectors. For example, retroviruses (in particular, lentiviral vectors) provide a convenient platform for gene delivery systems. A coding sequence of interest (for example, a sequence useful for gene therapy applications) can be inserted into a gene delivery vector and packaged in retroviral particles using techniques known in the art. Recombinant virus can then be isolated and delivered to cells of the subject either in vivo or ex vivo.

In one illustrative embodiment, retroviruses provide a convenient and effective platform for gene delivery systems. A selected nucleotide sequence that encodes an antigen corresponding to the target antigen can be inserted into a vector and packaged in retroviral particles using techniques known in the art. The recombinant virus can then be isolated and delivered to a subject. Several illustrative retroviral systems have been described examples of which include: U.S. Pat. No. 5,219,740; Miller and Rosman, 1989, Bio Techniques 7: 980-990; Miller, A. D., 1990, Human Gene Therapy 1: 5-14; Scarpa et al., 1991, Virology 180: 849-852; Burns et al., 1993, Proc. Natl. Acad. Sci. USA 90: 8033-8037; and Boris-Lawrie and Temin, 1993, Cur. Opin. Genet. Develop. 3: 102-109).

In addition, several illustrative adenovirus-based systems have also been described. Unlike retroviruses which integrate into the host genome, adenoviruses persist extrachromosomally thus minimizing the risks associated with insertional mutagenesis (see, e.g., Haj-Ahmad and Graham, 1986, J. Virol. 57: 267-274; Bett et al., 1993, J. Virol. 67: 5911-5921; Mittereder et al., 1994, Human Gene Therapy 5: 717-729; Seth et al., 1994, J. Virol. 68: 933-940; Barr et al., 1994, Gene Therapy 1: 51-58; Berkner, K. L., 1988, Bio Techniques 6: 616-629; and Rich et al., 1993, Human Gene Therapy 4: 461-476).

Various adeno-associated virus (AAV) vector systems have also been developed for polynucleotide delivery. AAV vectors can be readily constructed using techniques well known in the art. See, e.g., U.S. Pat. Nos. 5,173,414 and 5,139,941; International Publication Nos. WO 92/01070 and WO 93/03769; Lebkowski et al., 1988, Molec. Cell. Biol. 8: 3988-3996; Vincent et al., 1990, Vaccines 90, Cold Spring Harbor Laboratory Press; Carter, B. J., 1992, Current Opinion in Biotechnology 3: 533-539; Muzyczka, N., 1992, Current Topics in Microbiol. and Immunol. 158: 97-129; Kotin, R. M., 1994, Human Gene Therapy 5: 793-801; Shelling and Smith, 1994, Gene Therapy 1: 165-169; and Zhou et al., 1994, J. Exp. Med. 179: 1867-1875.

Additional viral vectors useful for delivering the antigen-encoding polynucleotide by gene transfer include those derived from the pox family of viruses, such as vaccinia virus and avian poxvirus. By way of example, vaccinia virus recombinants expressing an antigen-encoding polynucleotide can be constructed as follows. The DNA encoding a polypeptide is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the polypeptide of interest into the viral genome. The resulting TK⁽⁻⁾ recombinant can be selected by culturing the cells in the presence of 5-BrdU and picking viral plaques resistant thereto.

Alternatively, avipoxviruses, such as the fowlpox and canarypox viruses, can also be used to deliver the coding sequences of interest. The use of an Avipox vector is particularly desirable in human and other mammalian species since members of the Avipox genus can only productively replicate in susceptible avian species and therefore are not infective in mammalian cells. Methods for producing recombinant Avipoxviruses are known in the art and employ genetic recombination, as described above with respect to the production of vaccinia viruses. See, e.g., WO 91/12882; WO 89/03429; and WO 92/03545.

Any of a number of alphavirus vectors can also be used for delivery of polynucleotide compositions of the present invention, such as those vectors described in U.S. Pat. Nos. 5,843,723; 6,015,686; 6,008,035 and 6,015,694. Certain vectors based on Venezuelan Equine Encephalitis (VEE) can also be used, illustrative examples of which can be found in U.S. Pat. Nos. 5,505,947 and 5,643,576.

Moreover, molecular conjugate vectors, such as the adenovirus chimeric vectors described in Michael et al., J. Biol. Chem. 268:6866-69, 1993; and Wagner et al., Proc. Natl. Acad. Sci. USA 89:6099-6103, 1992, can also be used for gene delivery under the invention.

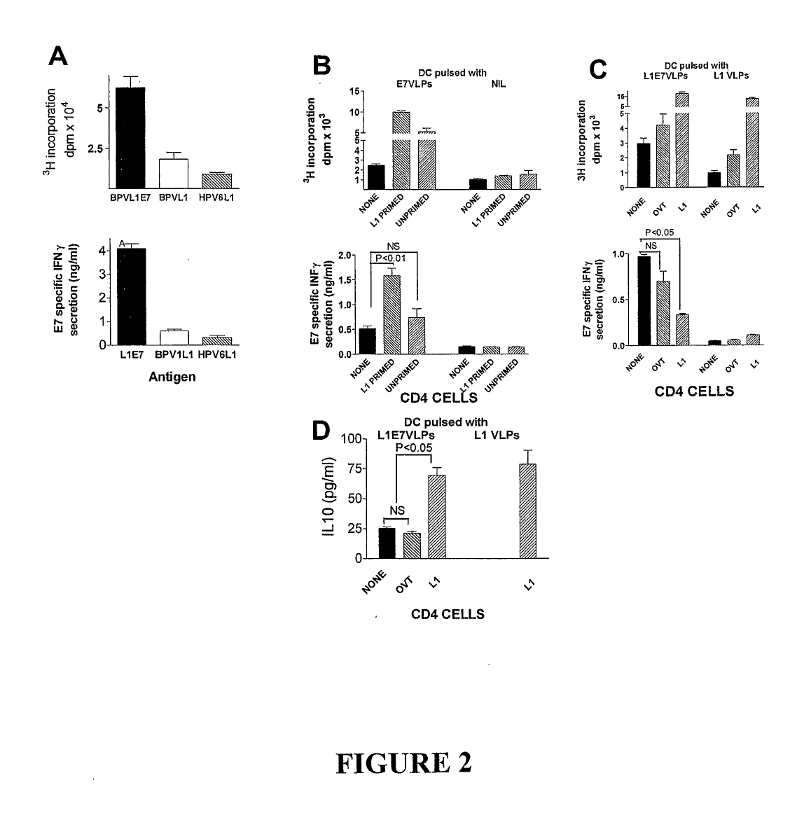

In other illustrative embodiments, lentiviral vectors are employed to deliver an antigen-encoding polynucleotide into selected cells or tissues. Typically, these vectors comprise a 5′ lentiviral LTR, a tRNA binding site, a packaging signal, a promoter operably linked to one or more genes of interest, an origin of second strand DNA synthesis and a 3′ lentiviral LTR, wherein the lentiviral vector contains a nuclear transport element. The nuclear transport element may be located either upstream (5′) or downstream (3′) of a coding sequence of interest (for example, a synthetic Gag or Env expression cassette of the present invention). A wide variety of lentiviruses may be utilized within the context of the present invention, including for example, lentiviruses selected from the group consisting of HIV, HIV-1, HIV-2, FIV, BIV, EIAV, MVV, CAEV, and SUV. Illustrative examples of lentiviral vectors are described in PCT Publication Nos. WO 00/66759, WO 00/00600, WO 99/24465, WO 98/51810, WO 99/51754, WO 99/31251, WO 99/30742, and WO 99/15641. Desirably, a third generation SIN lentivirus is used. Commercial suppliers of third generation SIN (self-inactivating) lentiviruses include Invitrogen (ViraPower Lentiviral Expression System). Detailed methods for construction, transfection, harvesting, and use of lentiviral vectors are given, for example, in the Invitrogen technical manual “ViraPower Lentiviral Expression System version B 050102 25-0501”, available at http://www.invitrogen.com/Content/Tech-Online/molecular_biology/manuals_p-ps/virapower_lentiviral_system_man.pdf. Lentiviral vectors have emerged as an efficient method for gene transfer. Improvements in biosafety characteristics have made these vectors suitable for use at biosafety level 2 (BL2). A number of safety features are incorporated into third generation SIN (self-inactivating) vectors. Deletion of the viral 3′ LTR U3 region results in a provirus that is unable to transcribe a full length viral RNA. In addition, a number of essential genes are provided in trans, yielding a viral stock that is capable of but a single round of infection and integration. Lentiviral vectors have several advantages, including: 1) pseudotyping of the vector using amphotropic envelope proteins allows them to infect virtually any cell type; 2) gene delivery to quiescent, post mitotic, differentiated cells, including neurons, has been demonstrated; 3) their low cellular toxicity is unique among transgene delivery systems; 4) viral integration into the genome permits long term transgene expression; 5) their packaging capacity (6-14 kb) is much larger than other retroviral, or adeno-associated viral vectors. In a recent demonstration of the capabilities of this system, lentiviral vectors expressing GFP were used to infect murine stem cells resulting in live progeny, germline transmission, and promoter-, and tissue-specific expression of the reporter (Ailles, L. E. and Naldini, L., HIV-1-Derived Lentiviral Vectors. In: Trono, D. (Ed.), Lentiviral Vectors, Springer-Verlag, Berlin, Heidelberg, New York, 2002, pp. 31-52). An example of the current generation vectors is outlined in FIG. 2 of a review by Lois et al. (Lois, C., Hong, E. J., Pease, S., Brown, E. J., and Baltimore, D., Germline transmission and tissue-specific expression of transgenes delivered by lentiviral vectors, Science, 295 (2002) 868-872).

In certain embodiments, a polynucleotide may be integrated into the genome of a target cell. This integration may be in the specific location and orientation via homologous recombination (gene replacement) or it may be integrated in a random, non-specific location (gene augmentation). In yet further embodiments, the polynucleotide may be stably maintained in the cell as a separate, episomal segment of DNA. Such polynucleotide segments or “episomes” encode sequences sufficient to permit maintenance and replication independent of or in synchronization with the host cell cycle. The manner in which the expression construct is delivered to a cell and where in the cell the polynucleotide remains is dependent on the type of expression construct employed.

In other embodiments, a polynucleotide is administered/delivered as “naked” DNA, for example as described in Ulmer et al., Science 259:1745-49, 1993 and reviewed by Cohen, Science 259:1691-92, 1993. The uptake of naked DNA may be increased by coating the DNA onto biodegradable beads, which are efficiently transported into the cells.

In still other embodiments, a composition of the present invention can be delivered via a particle bombardment approach, many of which have been described. In one illustrative example, gas-driven particle acceleration can be achieved with devices such as those manufactured by Powderject Pharmaceuticals PLC (Oxford, UK) and Powderject Vaccines Inc. (Madison, Wis.), some examples of which are described in U.S. Pat. Nos. 5,846,796; 6,010,478; 5,865,796; 5,584,807; and EP Patent No. 0500 799. This approach offers a needle-free delivery approach wherein a dry powder formulation of microscopic particles, such as polynucleotide or polypeptide particles, are accelerated to high speed within a helium gas jet generated by a hand held device, propelling the particles into a target tissue of interest.

In a related embodiment, other devices and methods that may be useful for gas-driven needle-less injection of compositions of the present invention include those provided by Bioject, Inc. (Portland, Oreg.), some examples of which are described in U.S. Pat. Nos. 4,790,824; 5,064,413; 5,312,335; 5,383,851; 5,399,163; 5,520,639 and 5,993,412.

2.2.2 Immune-Modulating Cell Embodiments

Antigen-Presenting Cells

The present invention also contemplates the use of antigen-presenting cells, which present an antigen corresponding to at least a portion of the target antigen, in the compositions of the present invention. Such antigen-presenting cells include professional or facultative antigen-presenting cells. Professional antigen-presenting cells function physiologically to present antigen in a form that is recognized by specific T cell receptors so as to stimulate or anergize a T lymphocyte or B lymphocyte mediated immune response. Professional antigen-presenting cells not only process and present antigens in the context of the major histocompatibility complex (MHC), but also possess the additional immunoregulatory molecules required to complete T cell activation or induce a tolerogenic response. Professional antigen-presenting cells include, but are not limited to, macrophages, monocytes, B lymphocytes, cells of myeloid lineage, including monocytic-granulocytic-DC precursors, marginal zone Kupffer cells, microglia, T cells, Langerhans cells and dendritic cells including interdigitating dendritic cells and follicular dendritic cells. Non-professional or facultative antigen-presenting cells typically lack one or more of the immunoregulatory molecules required to complete T lymphocyte activation or anergy. Examples of non-professional or facultative antigen-presenting cells include, but are not limited to, activated T lymphocytes, eosinophils, keratinocytes, astrocytes, follicular cells, microglial cells, thymic cortical cells, endothelial cells, Schwann cells, retinal pigment epithelial cells, myoblasts, vascular smooth muscle cells, chondrocytes, enterocytes, thymocytes, kidney tubule cells and fibroblasts. In some embodiments, the antigen-presenting cell is selected from monocytes, macrophages, B lymphocytes, cells of myeloid lineage, dendritic cells or Langerhans cells. In certain advantageous embodiments, the antigen-presenting cell expresses CD11c and includes a dendritic cell.

Antigen-presenting cells for stimulating an immune response to an antigen or group of antigens may be prepared according to any suitable method known to the skilled practitioner. Illustrative methods for preparing antigen-presenting cells for stimulating antigen-specific immune responses are described by Albert et al. (International Publication WO 99/42564), Takamizawa et al (1997, J Immunol, 158(5): 2134-2142), Thomas and Lipsky (1994, J Immunol, 153(9):4016-4028), O'Doherty et al. (1994, immunology, 82(3):487-93), Fearnley et al. (1997, Blood, 89(10): 3708-3716), Weissman et al. (1995, Proc Natl Acad Sci USA, 92(3):826-830), Freudenthal and Steinman (1990, Proc Natl Acad Sci USA, 87(19):7698-7702), Romani et al. (1996, J Immunol Methods, 196(2): 137-151), Reddy et al. (1997, Blood, 90(9):3640-3646), Thurnher et al. (1997, Exp Hematol, 25(3):232-237), Caux et al. (1996, J Exp Med, 184(2):695-706; 1996, Blood, 87(6):2376-85), Luft et al. (1998, Exp Hematol, 26(6):489-500; 1998, J Immunol, 161(4):1947-1953), Cella et al. (1999, J Exp Med, 189(5): 821-829; 1997, Nature, 388(644):782-787; 1996, J Exp Med, 184(2):747-572), Ahonen et al. (1999, Cell Immunol, 197(1):62-72) and Piemonti et al. (1999, J Immunol, 162(11):6473-6481).

In some embodiments, the antigen-presenting cells are isolated from a host, treated and then re-introduced or reinfused into the host. Conveniently, antigen-presenting cells can be obtained from the host to be treated either by surgical resection, biopsy, blood sampling, or other suitable technique. Such cells are referred to herein as “autologous” cells. In other embodiments, the antigen-presenting cells or cell lines are prepared and/or cultured from a different source than the host. Such cells are referred to herein as “allogeneic” cells. Desirably, allogeneic antigen-presenting cells or cell lines will share major and/or minor histocompatibility antigens to potential recipients (also referred to herein as ‘generic’ antigen-presenting cells or cell lines). In certain advantageous embodiments of this type, the generic antigen-presenting cells or cell lines comprise major histocompatibility (MHC) class I antigens compatible with a high percentage of the population (i.e., at least 10, 20, 30, 40, 50, 60, 70, 75, 80, 85, 90, 92, 94 or 98%) that is susceptible or predisposed to a particular condition. Suitably, the generic antigen-presenting cells or cell lines naturally express an immunostimulatory molecule as described herein, especially an immunostimulatory membrane molecule, at levels sufficient to trigger an immune response, desirably a T lymphocyte immune response (e.g., a cytotoxic T lymphocyte immune response), in the intended host. In certain embodiments, the antigen-presenting cells or cell lines are highly susceptible to treatment with at least one IFN as described in International Publication No. WO 01/88097 (i.e., implied high level expression of class I HLA).

In some embodiments, antigen-presenting cells are made antigen-specific by a process including contacting or ‘pulsing’ the antigen-presenting cells with an antigen that corresponds to at least a portion of the target antigen for a time and under conditions sufficient to permit the antigen to be internalized by the antigen-presenting cells; and culturing the antigen-presenting cells so contacted for a time and under conditions sufficient for the antigen to be processed for presentation by the antigen-presenting cells. The pulsed cells can then be used to stimulate autologous or allogeneic T cells in vitro or in vivo. The amount of antigen to be placed in contact with antigen-presenting cells can be determined empirically by persons of skill in the art. Typically antigen-presenting cells are incubated with antigen for about 1 to 6 hr at 37° C. Usually, for purified antigens and peptides, 0.1-10 μg/mL is suitable for producing antigen-specific antigen-presenting cells. The antigen should be exposed to the antigen-presenting cells for a period of time sufficient for those cells to internalize the antigen. The time and dose of antigen necessary for the cells to internalize and present the processed antigen may be determined using pulse-chase protocols in which exposure to antigen is followed by a washout period and exposure to a read-out system e.g., antigen reactive T cells. Once the optimal time and dose necessary for cells to express processed antigen on their surface is determined, a protocol may be used to prepare cells and antigen for inducing tolerogenic responses. Those of skill in the art will recognize in this regard that the length of time necessary for an antigen-presenting cell to present an antigen may vary depending on the antigen or form of antigen employed, its dose, and the antigen-presenting cell employed, as well as the conditions under which antigen loading is undertaken. These parameters can be determined by the skilled artisan using routine procedures.

The delivery of exogenous antigen to an antigen-presenting cell can be enhanced by methods known to practitioners in the art. For example, several different strategies have been developed for delivery of exogenous antigen to the endogenous processing pathway of antigen-presenting cells, especially dendritic cells. These methods include insertion of antigen into pH-sensitive liposomes (Zhou and Huang, 1994, Immunomethods, 4:229-235), osmotic lysis of pinosomes after pinocytic uptake of soluble antigen (Moore et al., 1988, Cell, 54:777-785), coupling of antigens to potent adjuvants (Aichele et al., 1990, J. Exp. Med., 171: 1815-1820; Gao et al., 1991, J. Immunol, 147: 3268-3273; Schulz et al., 1991, Proc. Natl. Acad. Sci. USA, 88: 991-993; Kuzu et al., 1993, Euro. J. Immunol., 23: 1397-1400; and Jondal et al., 1996, Immunity 5: 295-302) and apoptotic cell delivery of antigen (Albert et al. 1998, Nature 392:86-89; Albert et al. 1998, Nature Med. 4:1321-1324; and in International Publications WO 99/42564 and WO 01/85207). Recombinant bacteria (eg. E. coli) or transfected host mammalian cells may be pulsed onto dendritic cells (as particulate antigen, or apoptotic bodies respectively) for antigen delivery. Recombinant chimeric virus-like particles (VLPs) have also been used as vehicles for delivery of exogenous heterologous antigen to the MHC class I processing pathway of a dendritic cell line (Bachmann et al., 1996, Eur. J. Immunol., 26(11): 2595-2600).

Alternatively, or in addition, an antigen may be linked to, or otherwise associated with, a cytolysin to enhance the transfer of the antigen into the cytosol of an antigen-presenting cell of the invention for delivery to the MHC class I pathway. Exemplary cytolysins include saponin compounds such as saponin-containing Immune Stimulating Complexes (ISCOMs) (see e.g., Cox and Coulter, 1997, Vaccine 15(3): 248-256 and U.S. Pat. No. 6,352,697), phospholipases (see, e.g., Camilli et al., 1991, J. Exp. Med. 173: 751-754), pore-forming toxins (e.g., an α-toxin), natural cytolysins of gram-positive bacteria, such as listeriolysin O (LLO, e.g., Mengaud et al., 1988, Infect. Immun. 56: 766-772 and Portnoy et al., 1992, Infect. Immun. 60: 2710-2717), streptolysin O (SLO, e.g., Palmer et al., 1998, Biochemistry 37(8): 2378-2383) and perfringolysin O (PFO, e.g., Rossjohn et al., Cell 89(5): 685-692). Where the antigen-presenting cell is phagosomal, acid activated cytolysins may be advantageously used. For example, listeriolysin exhibits greater pore-forming ability at mildly acidic pH (the pH conditions within the phagosome), thereby facilitating delivery of vacuole (including phagosome and endosome) contents to the cytoplasm (see, e.g., Portnoy et al., Infect. Immun. 1992, 60: 2710-2717).

The cytolysin may be provided together with a pre-selected antigen in the form of a single composition or may be provided as a separate composition, for contacting the antigen-presenting cells. In one embodiment, the cytolysin is fused or otherwise linked to the antigen, wherein the fusion or linkage permits the delivery of the antigen to the cytosol of the target cell. In another embodiment, the cytolysin and antigen are provided in the form of a delivery vehicle such as, but not limited to, a liposome or a microbial delivery vehicle selected from virus, bacterium, or yeast. Suitably, when the delivery vehicle is a microbial delivery vehicle, the delivery vehicle is non-virulent. In a preferred embodiment of this type, the delivery vehicle is a non-virulent bacterium, as for example described by Portnoy et al. in U.S. Pat. No. 6,287,556, comprising a first polynucleotide encoding a non-secreted functional cytolysin operably linked to a regulatory polynucleotide which expresses the cytolysin in the bacterium, and a second polynucleotide encoding one or more pre-selected antigens. Non-secreted cytolysins may be provided by various mechanisms, e.g., absence of a functional signal sequence, a secretion incompetent microbe, such as microbes having genetic lesions (e.g., a functional signal sequence mutation), or poisoned microbes, etc. A wide variety of nonvirulent, non-pathogenic bacteria may be used; preferred microbes are relatively well characterized strains, particularly laboratory strains of E. coli, such as MC4100, MC1061, DH5α, etc. Other bacteria that can be engineered for the invention include well-characterized, nonvirulent, non-pathogenic strains of Listeria monocytogenes, Shigella flexneri, mycobacterium, Salmonella, Bacillus subtilis, etc. In particular embodiments, the bacteria are attenuated to be non-replicative, non-integrative into the host cell genome, and/or non-motile inter- or intra-cellularly.

The delivery vehicles described above can be used to deliver one or more antigens to virtually any antigen-presenting cell capable of endocytosis of the subject vehicle, including phagocytic and non-phagocytic antigen-presenting cells. In embodiments when the delivery vehicle is a microbe, the subject methods generally require microbial uptake by the target cell and subsequent lysis within the antigen-presenting cell vacuole (including phagosomes and endosomes).

In other embodiments, the antigen is produced inside the antigen-presenting cell by introduction of a suitable expression vector as for example described above. The antigen-encoding portion of the expression vector may comprise a naturally-occurring sequence or a variant thereof, which has been engineered using recombinant techniques. In one example of a variant, the codon composition of an antigen-encoding polynucleotide is modified to permit enhanced expression of the antigen in a target cell or tissue of choice using methods as set forth in detail in International Publications WO 99/02694 and WO 00/42215. Briefly, these methods are based on the observation that translational efficiencies of different codons vary between different cells or tissues and that these differences can be exploited, together with codon composition of a gene, to regulate expression of a protein in a particular cell or tissue type. Thus, for the construction of codon-optimized polynucleotides, at least one existing codon of a parent polynucleotide is replaced with a synonymous codon that has a higher translational efficiency in a target cell or tissue than the existing codon it replaces. Although it is preferable to replace all the existing codons of a parent nucleic acid molecule with synonymous codons which have that higher translational efficiency, this is not necessary because increased expression can be accomplished even with partial replacement. Suitably, the replacement step affects 5, 10, 15, 20, 25, 30%, more suitably 35, 40, 50, 60, 70% or more of the existing codons of a parent polynucleotide.

The expression vector for introduction into the antigen-presenting cell will be compatible therewith such that the antigen-encoding polynucleotide is expressible by the cell. For example, expression vectors of this type can be derived from viral DNA sequences including, but not limited to, adenovirus, adeno-associated viruses, herpes-simplex viruses and retroviruses such as B, C, and D retroviruses as well as spumaviruses and modified lentiviruses. Suitable expression vectors for transfection of animal cells are described, for example, by Wu and Ataai (2000, Curr. Opin. Biotechnol. 11(2):205-208), Vigna and Naldini (2000, J. Gene Med. 2(5):308-316), Kay, et al. (2001, Nat. Med. 7(1):33-40), Athanasopoulos, et al. (2000, Int. J. Mol. Med. 6(4):363-375) and Walther and Stein (2000, Drugs 60(2):249-271). The expression vector is introduced into the antigen-presenting cell by any suitable means which will be dependent on the particular choice of expression vector and antigen-presenting cell employed. Such means of introduction are well-known to those skilled in the art. For example, introduction can be effected by use of contacting (e.g., in the case of viral vectors), electroporation, transformation, transduction, conjugation or triparental mating, transfection, infection membrane fusion with cationic lipids, high-velocity bombardment with DNA-coated microprojectiles, incubation with calcium phosphate-DNA precipitate, direct microinjection into single cells, and the like. Other methods also are available and are known to those skilled in the art. Alternatively, the vectors are introduced by means of cationic lipids, e.g., liposomes. Such liposomes are commercially available (e.g., Lipofectin®, Lipofectamine™, and the like, supplied by Life Technologies, Gibco BRL, Gaithersburg, Md.). It will be understood by persons of skill in the art that the techniques for assembling and expressing antigen-encoding nucleic acid molecules, immunoregulatory molecules and/or cytokines as described herein e.g., synthesis of oligonucleotides, nucleic acid amplification techniques, transforming cells, constructing vectors, expressions system and the like and transducing or otherwise introducing nucleic acid molecules into cells are well established in the art, and most practitioners are familiar with the standard resource materials for specific conditions and procedures.

In some embodiments, the antigen-specific antigen-presenting cells are obtained by isolating antigen-presenting cells or their precursors from a cell population or tissue to which modification of an immune response is desired. Typically, some of the isolated antigen-presenting cells or precursors will constitutively present antigens or have taken up such antigen in vivo that are targets or potential targets of an immune response for which stimulation or inhibition of an immune response is desired. In this instance, the delivery of exogenous antigen is not essential. Alternatively, cells may be derived from biopsies of healthy or diseased tissues, lysed or rendered apoptotic and the pulsed onto antigen-presenting cells (e.g., dendritic cells). In certain embodiments of this type, the antigen-presenting cells represent cancer or tumor cells to which an antigen-specific immune response is required. Illustrative examples of cancers or tumor cells include cells of sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; myelomonocytic, monocytic and erythroleukemia); chronic leukemia (chronic myelocytic (granulocyte) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In certain embodiments, the cancer or tumor cells are selected from the group melanoma cells and mammary carcinoma cells.

In some of the above embodiments, the cancer or tumor cells will constitute facultative or non-professional antigen-presenting cells, and may in some instances require further modification to enhance their antigen-presenting functions. In these instances, the antigen-presenting cells are further modified to express one or more immunoregulatory molecules, which include any molecules occurring naturally in animals that may regulate or directly influence immune responses including: proteins involved in antigen processing and presentation such as TAP1/TAP2 transporter proteins, proteosome molecules such as LMP2 and LMP7, heat shock proteins such as gp96, HSP70 and HSP90, and major histocompatibility complex (MHC) or human leukocyte antigen (HLA) molecules; factors that provide co-stimulation signals for T cell activation such as B7 and CD40; factors that provide co-inhibitory signals for direct killing of T cells or induction of T lymphocyte or B lymphocyte anergy or stimulation of T regulatory cell (Treg) generation such as OX-2, programmed death-1 ligand (PD-1L); accessory molecules such as CD83; chemokines; lymphokines and cytokines such as IFN s α, β and γ, interleukins (e.g., IL-2, IL-7, IL-12, IL-15, IL-22, etc.), factors stimulating cell growth (e.g., GM-SCF) and other factors (e.g., tumor necrosis factors (TNFs), DC-SIGN, MIP1α, MIP1β and transforming growth factor-β (TGF-β). In certain advantageous embodiments, the immunoregulatory molecules are selected from a B7 molecule (e.g., B7-1, B7-2 or B7-3) and an ICAM molecule (e.g., ICAM-1 and ICAM-2).

Instead of recombinantly expressing immunoregulatory molecules, antigen-presenting cells expressing the desired immunostimulatory molecule(s) may be isolated or selected from a heterogeneous population of cells. Any method of isolation/selection is contemplated by the present invention, examples of which are known to those of skill in the art. For instance, one can take advantage of one or more particular characteristics of a cell to specifically isolate that cell from a heterogeneous population. Such characteristics include, but are not limited to, anatomical location of a cell, cell density, cell size, cell morphology, cellular metabolic activity, cell uptake of ions such as Ca²⁺, K⁺, and H⁺ ions, cell uptake of compounds such as stains, markers expressed on the cell surface, protein fluorescence, and membrane potential. Suitable methods that can be used in this regard include surgical removal of tissue, flow cytometry techniques such as fluorescence-activated cell sorting (FACS), immunoaffinity separation (e.g., magnetic bead separation such as Dynabead™ separation), density separation (e.g., metrizamide, Percoll™, or Ficol™ gradient centrifugation), and cell-type specific density separation. Desirably, the cells are isolated by flow cytometry or by immunoaffinity separation using an antigen-binding molecule that is immuno-interactive with the immunoregulatory molecule.

Alternatively, the immunoregulatory molecule can be provided to the antigen-presenting cells in soluble form. In some embodiments of this type, the immunoregulatory molecule is a B7 molecule that lacks a functional transmembrane domain (e.g., that comprises a B7 extracellular domain), non-limiting examples of which are described by McHugh et al. (1998, Clin. Immunol. Immunopathol. 87(1):50-59), Faas et al. (2000, J. Immunol. 164(12):6340-6348) and Jeannin et al. (2000, Immunity 13(3):303-312). In other embodiments of this type, the immunostimulatory protein is a B7 derivative including, but not limited to, a chimeric or fusion protein comprising a B7 molecule, or biologically active fragment thereof, or variant or derivative of these, linked together with an antigen binding molecule such as an immunoglobulin molecule or biologically active fragment thereof. For example, a polynucleotide encoding the amino acid sequence corresponding to the extracellular domain of the B7-1 molecule, containing amino acids from about position 1 to about position 215, is joined to a polynucleotide encoding the amino acid sequences corresponding to the hinge, CH2 and CH3 regions of human Ig Cγ1, using PCR, to form a construct that is expressed as a B7Ig fusion protein. DNA encoding the amino acid sequence corresponding to a B71g fusion protein has been deposited with the American Type culture Collection (ATCC) in Rockville, Md., under the Budapest Treaty on May 31, 1991 and accorded accession number 68627. Techniques for making and assembling such B7 derivatives are disclosed for example by Linsley et al. (U.S. Pat. No. 5,580,756). Reference also may be made to Sturmhoefel et al. (1999, Cancer Res. 59: 4964-4972) who disclose fusion proteins comprising the extracellular region of B7-1 or B7-2 fused in frame to the Fc portion of IgG2a.

The half-life of a soluble immunoregulatory molecule may be prolonged by any suitable procedure if desired. Preferably, such molecules are chemically modified with polyethylene glycol (PEG), including monomethoxy-polyethylene glycol, as for example disclosed by Chapman et al (1999, Nature Biotechnology 17: 780-783).

Alternatively, or in addition, the antigen-presenting cells are cultured in the presence of at least one IFN for a time and under conditions sufficient to enhance the antigen presenting function of the cells and washing the cells to remove the IFN (s). In certain advantageous embodiments of this type, the step of culturing may comprise contacting the cells with at least one type I IFN and/or a type II IFN. The at least one type I IFN is suitably selected from the group consisting of an IFN-α, an IFN-β, a biologically active fragment of an IFN-α, a biologically active fragment of an IFN-β, a variant of an IFN-α, a variant of an IFN-β, a variant of a said biologically active fragment, a derivative of an IFN-α, a derivative of an IFN-β, a derivative of a said biologically active fragment, a derivative of a said variant, an analogue of IFN-α and an analogue of IFN-β. Typically, the type II IFN is selected from the group consisting of an IFN-γ, a biologically active fragment of an IFN-γ, a variant of an IFN-γ, a variant of said biologically active fragment, a derivative of an IFN-γ, a derivative of said biologically active fragment, a derivative of said variant and an analogue of an IFN-γ. Exemplary methods and conditions for enhancing the antigen-presenting functions of antigen-presenting cells using IFN treatment are described in International Publication No. WO 01/88097.

In some embodiments, the antigen-presenting cells (e.g., cancer cells) or cell lines are suitably rendered inactive to prevent further proliferation once administered to the subject. Any physical, chemical, or biological means of inactivation may be used, including but not limited to irradiation (generally with at least about 5,000 cGy, usually at least about 10,000 cGy, typically at least about 20,000 cGy); or treatment with mitomycin-C (usually at least 10 μg/mL; more usually at least about 50 μg/mL).

The antigen-presenting cells may be obtained or prepared to contain and/or express one or more antigens by any number of means, such that the antigen(s) or processed form(s) thereof, is (are) presented by those cells for potential modulation of other immune cells, including T lymphocytes and B lymphocytes, and particularly for producing T lymphocytes and B lymphocytes that are primed to respond to a specified antigen or group of antigens.

Immune Effector Cells

In some embodiments, the antigen-presenting cells described above are useful for producing primed T lymphocytes to an antigen or group of antigens. The efficiency of inducing lymphocytes, especially T lymphocytes, to exhibit an immune response to a specified antigen can be determined by any suitable method including, but not limited to, assaying T lymphocyte cytolytic activity in vitro using for example antigen-specific antigen-presenting cells as targets of antigen-specific cytolytic T lymphocytes (CTL); assaying antigen-specific T lymphocyte proliferation (see, e.g., Vollenweider and Groseurth, 1992, J. Immunol. Meth. 149: 133-135), measuring B cell response to the antigen using, for example, ELISPOT assays, and ELISA assays; interrogating cytokine profiles; or measuring delayed-type hypersensitivity (DTH) responses by test of skin reactivity to a specified antigen (see, e.g., Chang et al. (1993, Cancer Res. 53: 1043-1050). Other methods known to practitioners in the art, which can detect the presence of antigen on the surface of antigen-presenting cells after exposure to the antigen, are also contemplated by the present invention.

Accordingly, the present invention also provides antigen-specific B or T lymphocytes, especially T lymphocytes, which respond in an antigen-specific fashion to representation of the antigen. In some embodiments, antigen-specific T lymphocytes are produced by contacting an antigen-presenting cell as defined above with a population of T lymphocytes, which may be obtained from any suitable source such as spleen or tonsil/lymph nodes but is preferably obtained from peripheral blood. The T lymphocytes can be used as crude preparations or as partially purified or substantially purified preparations, which are suitably obtained using standard techniques as, for example, described in “Immunochemical Techniques, Part G: Separation and Characterization of Lymphoid Cells” (Meth. in Enzymol. 108, Edited by Di Sabato et al, 1984, Academic Press). This includes rosetting with sheep red blood cells, passage across columns of nylon wool or plastic adherence to deplete adherent cells, immunomagnetic or flow cytometric selection using appropriate monoclonal antibodies is known in the art.

The preparation of T lymphocytes is contacted with antigen-specific antigen-presenting cells as described herein for an adequate period of time for priming the T lymphocytes to the antigen or antigens presented by those antigen-presenting cells. This period will usually be at least about 1 day, and up to about 5 days.

2.2.3 Antigen-Binding Molecules

The invention also contemplates the use of antigen-binding molecules that are specifically immuno-interactive with a selected target antigen as immune-modulating agents. In some embodiments, the target antigen is expressed in a disease or condition or by a specific pathogen for which an enhanced immune response is required. In other embodiments, the target antigen is aberrantly expressed, typically at a higher level in the disease or condition as compared to the normal state or to a state in which the disease or condition is absent. The antigen-binding molecule is suitably interactive with a target antigen as described for example in Section 2.2.1. Numerous antigen-binding molecule useful in the present invention are known in the art. In an illustrative example in which colon cancer is the subject of the treatment, the antigen-binding molecule is immuno-interactive with an antigen selected from the Cripto-1 protein, Pim-1 protein or an antigen present in a colon cancer cell lysate, as disclosed, for example, in United States Patent Application Publication No. 20040176576.

In some embodiments, the antigen-binding molecule is a whole polyclonal antibody. Such antibodies may be prepared, for example, by injecting an antigen that corresponds to at least a portion of the target antigen into a production species, which may include mice or rabbits, to obtain polyclonal antisera. Methods of producing polyclonal antibodies are well known to those skilled in the art. Exemplary protocols which may be used are described for example in Coligan et al., CURRENT PROTOCOLS IN IMMUNOLOGY, (John Wiley & Sons, Inc, 1991), and Ausubel et al., (1994-1998, supra), in particular Section III of Chapter 11.

In lieu of polyclonal antisera obtained in a production species, monoclonal antibodies may be produced using the standard method as described, for example, by Köhler and Milstein (1975, Nature 256, 495-497), or by more recent modifications thereof as described, for example, in Coligan et al., (1991, supra) by immortalizing spleen or other antibody producing cells derived from a production species which has been inoculated with one or more antigens as described above.

The invention also contemplates as antigen-binding molecules Fv, Fab, Fab′ and F(ab′)₂ immunoglobulin fragments. Alternatively, the antigen-binding molecule may comprise a synthetic stabilized Fv fragment. Exemplary fragments of this type include single chain Fv fragments (sFv, frequently termed scfv) in which a peptide linker is used to bridge the N terminus or C terminus of a V_{H} domain with the C terminus or N-terminus, respectively, of a V_{L} domain. ScFv lack all constant parts of whole antibodies and are not able to activate complement. ScFvs may be prepared, for example, in accordance with methods outlined in Kreber et al (Kreber et al. 1997, J. Immunol. Methods; 201(1): 35-55). Alternatively, they may be prepared by methods described in U.S. Pat. No. 5,091,513, European Patent No 239,400 or the articles by Winter and Milstein (1991, Nature 349:293) and Plückthun et al (1996, In Antibody engineering: A practical approach. 203-252). In another embodiment, the synthetic stabilized Fv fragment comprises a disulphide stabilized Fv (dsFv) in which cysteine residues are introduced into the V_{H} and V_{L} domains such that in the fully folded Fv molecule the two residues will form a disulphide bond between them. Suitable methods of producing dsFv are described for example in (Glockscuther et al. Biochem. 29: 1363-1367; Reiter et al. 1994, J. Biol. Chem. 269: 18327-18331; Reiter et al. 1994, Biochem. 33: 5451-5459; Reiter et al. 1994. Cancer Res. 54: 2714-2718; Webber et al. 1995, Mol. Immunol. 32: 249-258).

2.3 Ancillary Components

In some embodiments the composition further comprises one or more cytokines, which are suitably selected from flt3, SCF, IL-3, IL-6, GM-CSF, G-CSF, TNF-α, IL-4, TNF-β, LT-β, IL-2, IL-7, IL-9, IL-15, IL-13, IL-5, IL-1α, IL-1β, IFN-γ, IL-17, IL-16, IL-18, HGF, IL-11, MSP, FasL, TRAIL, TRANCE, LIGHT, TWEAK, CD27L, CD30L, CD40L, APRIL, TALL-1, 4-1BBL, OX40L, GITRL, IGF-I, IGF-II, HGF, MSP, FGF-a, FGF-b, FGF-3-19, NGF, BDNF, NTs, Tpo, Epo, Angl-4, PDGF-AA, PDGF-BB, VEGF-A, VEGF-B, VEGF-C, VEGF-D, P1GF, EGF, TGF-α, AR, BTC, HRGs, HB-EGF, SMDF, OB, CT-1, CNTF, OSM, SCF, Flt-3L, M-CSF, MK and PTN or their functional, recombinant or chemical equivalents or homologues thereof. Preferably the cytokine is selected from the group consisting of IL-12, IL-3, IL-5, TNF, GMCSF, and IFN-γ.

### 3. Cell Based Therapy or Prophylaxis

In accordance with the present invention, an inhibitor of Il-10 function, as described for example in Section 2.1, can be administered to a patient, together with antigen-presenting cells and/or immune effector cells as described in Section 2.2.2 for priming or boosting an immune response. These cell based compositions are useful, therefore, for treating or preventing a disease or condition that is associated with the presence or aberrant expression of a target antigen. The cells of the invention can be introduced into a patient by any means (e.g., injection), which produces the desired immune response to an antigen or group of antigens. The cells may be derived from the patient (i.e., autologous cells) or from an individual or individuals who are MHC matched or mismatched (i.e., allogeneic) with the patient. Typically, autologous cells are injected back into the patient from whom the source cells were obtained. The injection site may be subcutaneous, intraperitoneal, intramuscular, intradermal, or intravenous. The cells may be administered to a patient already suffering from a disease or condition or who is predisposed to a disease or condition in sufficient number to treat or prevent or alleviate the symptoms of the disease or condition. The number of cells injected into the patient in need of the treatment or prophylaxis may vary depending on inter alia, the antigen or antigens and size of the individual. This number may range for example between about 10³ and 10¹¹, and usually between about 10⁵ and 10⁷ cells (e.g., dendritic cells or T lymphocytes). Single or multiple administrations of the cells can be carried out with cell numbers and pattern being selected by the treating physician. The cells should be administered in a pharmaceutically acceptable carrier, which is non-toxic to the cells and the individual. Such carrier may be the growth medium in which the cells were grown, or any suitable buffering medium such as phosphate buffered saline. The cells may be administered alone or as an adjunct therapy in conjunction with other therapeutics known in the art for the treatment or prevention of unwanted immune responses for example but not limited to glucocorticoids, methotrexate, D-penicillamine, hydroxychloroquine, gold salts, sulfasalazine, TNFα or interleukin-1 inhibitors, and/or other forms of specific immunotherapy.

### 4. Pharmaceutical Formulations

The present invention also contemplates immunomodulating formulations, including vaccines, comprising an inhibitor of IL-10 function, as described for example in Section 2.1, and an immune-stimulating agent e.g., an antigen as described in Sections 2.2.1, an immune effector cell as described in Section 2.2.2 or an antigen-binding molecule as described in Section 2.2.3, or combinations thereof (therapeutic/prophylactic agents) as active ingredients for the treatment or prophylaxis of various diseases or conditions associated with the presence or aberrant expression of a target antigen. These therapeutic/prophylactic agents can be administered to a patient either by themselves, or in formulations where they are mixed with a suitable pharmaceutically acceptable carrier and/or diluent, or an adjuvant.

The preparation of such formulations uses routine methods known to persons skilled in the art. Typically, such formulations and vaccines are prepared as injectables, either as liquid solutions or suspensions; solid forms suitable for solution in, or suspension in, liquid prior to injection may also be prepared. The preparation may also be emulsified. The active immunogenic ingredients are often mixed with excipients that are pharmaceutically acceptable and compatible with the active ingredient. Suitable excipients are, for example, water, phosphate buffered saline, saline, dextrose, glycerol, ethanol, or the like and combinations thereof. In addition, if desired, the vaccine may contain minor amounts of auxiliary substances such as wetting or emulsifying agents, pH buffering agents, and/or adjuvants that enhance the effectiveness of the vaccine. Examples of adjuvants which may be effective include but are not limited to: surface active substances such as hexadecylamine, octadecylamine, octadecyl amino acid esters, lysolecithin, dimethyldioctadecylammonium bromide, N,N-dicoctadecyl-N′,N′bis(2-hydroxyethyl-propanediamine), methoxyhexadecylglycerol, and pluronic polyols; polyamines such as pyran, dextransulfate, poly IC carbopol; mineral gels such as aluminum phosphate, aluminum hydroxide or alum; peptides such as muramyl dipeptide and derivatives such as N-acetyl-muramyl-L-threonyl-D-isoglutamine (thur-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 1983A, referred to as MTP-PE), and RIBI, which contains three components extracted from bacteria, monophosphoryl lipid A, dimethylglycine, tuftsin; oil emulsions; trehalose dimycolate and cell wall skeleton (MPL+TDM+CWS) in a 2% squalene/Tween 80 emulsion; lymphokines; QuilA and immune stimulating complexes (ISCOMS). For example, the effectiveness of an adjuvant may be determined by measuring the amount of antibodies resulting from the administration of the vaccine, wherein those antibodies are directed against one or more antigens presented by the treated cells of the vaccine.

The active ingredients should be administered in a pharmaceutically acceptable carrier, which is non-toxic to the cells and the individual to be treated. Such carrier may be the growth medium in which the cells were grown. Compatible excipients include isotonic saline, with or without a physiologically compatible buffer like phosphate or Hepes and nutrients such as dextrose, physiologically compatible ions, or amino acids, and various culture media suitable for use with cell populations, particularly those devoid of other immunogenic components. Carrying reagents, such as albumin and blood plasma fractions and nonactive thickening agents, may also be used. Non-active biological components, to the extent that they are present in the vaccine, are preferably derived from a syngeneic animal or human as that to be treated, and are even more preferably obtained previously from the subject. The injection site may be subcutaneous, intraperitoneal, intramuscular, intradermal, or intravenous.

If soluble actives are employed, the soluble active ingredients can be formulated into the vaccine as neutral or salt forms. Pharmaceutically acceptable salts include the acid addition salts (formed with free amino groups of the peptide) and which are formed with inorganic acids such as, for example, hydrochloric or phosphoric acids, or such organic acids such as acetic, oxalic, tartaric, maleic, and the like. Salts formed with the free carboxyl groups may also be derived from inorganic basis such as, for example, sodium, potassium, ammonium, calcium, or ferric hydroxides, and such organic basis as isopropylamine, trimethylamine, 2-ethylamino ethanol, histidine, procaine, and the like.

If desired, devices or pharmaceutical compositions or compositions containing the vaccine and suitable for sustained or intermittent release could be, in effect, implanted in the body or topically applied thereto for the relatively slow release of such materials into the body.

Techniques for formulation and administration may be found in “Remington's Pharmaceutical Sciences,” Mack Publishing Co., Easton, Pa., latest edition. Suitable routes may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections.

The dosage to be administered may depend on the subject to be treated inclusive of the age, sex, weight and general health condition thereof. The dosage will also take into consideration the binding affinity of the inhibitor of L-10 function to its target molecule, the immunogenicity of the immune stimulator, their bioavailability and their in vivo and pharmacokinetic properties. In this regard, precise amounts of the agent(s) for administration can also depend on the judgment of the practitioner. In determining the effective amount of the agent(s) to be administered in the treatment of a disease or condition, the physician or veterinarian may evaluate the progression of the disease or condition over time. In any event, those of skill in the art may readily determine suitable dosages of the agents of the invention without undue experimentation. Cell-containing compositions and vaccines are suitably administered to a patient in the range of between about 10⁴ and 10¹⁰, and more preferably between about 10⁶ and 10⁸ treated cells/administration. The dosage of the actives administered to a patient should be sufficient to effect a beneficial response in the patient over time such as a reduction in the symptoms associated with the cancer or tumor. For example usual patient dosages for systemic administration of inhibitors of IL-10 function or polypeptide antigens range from about 0.1-200 g, typically from about 1-160 g and more typically from about 10-70 g. Stated in terms of patient body weight, usual dosages range from about 1.5-3000 mg/kg, typically from about 15-2500 mg/kg, more typically from about 150-1000 mg/kg and even more typically from about 20-50 mg/kg. The dosages may be administered at suitable intervals to maintain IL-10 inhibition or to maintain or boost the immune response against a target antigen. Such intervals can be ascertained using routine procedures known to persons of skill in the art and can vary depending on the type of active agent employed and its formulation. For example, the interval may be daily, every other day, weekly, fortnightly, monthly, bimonthly, quarterly, half-yearly or yearly.

Thus, the inhibitor of IL-10 function and the immune stimulator may be provided in effective amounts to stimulate or enhance the immune response to a target antigen. This process may involve administering the inhibitor of IL-10 function separately, simultaneously or sequentially with the immune stimulator. In some embodiments, this may be achieved by administering a single composition or pharmacological formulation that includes both agents, or by administering two separate compositions or formulations at the same time, wherein one composition includes the inhibitor of IL-10 function and the other, the immune stimulator. In other embodiments, the treatment with the inhibitor of IL-10 function may precede or follow the treatment with the immune stimulator by intervals ranging from minutes to days. In embodiments where the inhibitor of IL-10 function is applied separately to the immune stimulator, one would generally ensure that a significant period of time did not expire between the time of each delivery, such that the inhibitor of IL-10 function would still be able to exert an advantageously combined effect on the immune system with the immune stimulator, in particular, to maintain or enhance a subject's capacity to mount an antigen-specific CD8⁺ IFN-γ-producing immune response upon subsequent challenge with the immune stimulator. In such instances, it is contemplated that one would contact the cell with both modalities within about 1-12 hours of each other and, more suitably, within about 2-6 hours of each other. In some situations, it may be desirable to extend the time period for treatment significantly, however, where several hours (2, 3, 4, 5, 6 or 7) to several days (1, 2, 3, 4, 5, 6, 7 or 8) lapse between the respective administrations.

As indicated above, it is conceivable that more than one administration of either the inhibitor of IL-10 function or immune stimulator will be desired. Various combinations may be employed, where the inhibitor of IL-10 function is “A” and the immune stimulator is “B”, as exemplified below:

A/B/A B/A/B B/B/A A/A/B B/A/A A/B/B B/B/B/A
B/B/A/B A/A/B/B A/B/A/B A/B/B/A B/B/A/A B/A/B/A
B/A/A/B B/B/B/A A/A/A/B B/A/A/A A/B/A/A A/A/B/A
A/B/B/B B/A/B/B B/B/A/B.

Other combinations are contemplated. Again, both agents are delivered to a subject's immune system in a combined amount effective to maintain or enhance a subject's capacity to mount an antigen-specific CD8⁺ IFN-γ-producing immune response upon subsequent challenge with the immune stimulator.

### 5. Methods for Modulating Immune Responses

The compositions of the invention may be used for stimulating an immune response to a target antigen in a subject that is immunologically naïve to the target antigen or that has previously raised an immune response to that antigen. Thus, the present invention also extends to methods for enhancing an immune response in a subject by administering to the subject the compositions or vaccines of the invention. Advantageously, the immune response is a cell-mediated immune response (e.g., a T-cell mediated response, which desirably includes CD8⁺ IFN-γ-producing T cells). The active ingredients of the compositions may be administered either sequentially, simultaneously or separately, as discussed for example above.

Also encapsulated by the present invention is a method for treatment and/or prophylaxis of a disease or condition, comprising administering to a patient in need of such treatment an effective amount of a inhibitor of IL-10 function, together with an effective amount of an immune stimulator, as broadly described above. In certain embodiments, the target antigen is associated with or responsible for a disease or condition which is suitably selected from cancers, infectious diseases and diseases characterized by immunodeficiency. Examples of cancer include but are not limited to ABL1 protooncogene, AIDS related cancers, acoustic neuroma, acute lymphocytic leukemia, acute myeloid leukemia, adenocystic carcinoma, adrenocortical cancer, agnogenic myeloid metaplasia, alopecia, alveolar soft-part sarcoma, anal cancer, angiosarcoma, aplastic anemia, astrocytoma, ataxia telangiectasia, basal cell carcinoma (skin), bladder cancer, bone cancers, bowel cancer, brain stem glioma, brain and CNS tumors, breast cancer, CNS tumors, carcinoid tumors, cervical cancer, childhood brain tumors, childhood cancer, childhood leukemia, childhood soft tissue sarcoma, chondrosarcoma, choriocarcinoma, chronic lymphocytic leukemia, chronic myeloid leukemia, colorectal cancers, cutaneous T-cell lymphoma, dermatofibrosarcoma protuberans, desmoplastic small round cell tumor, ductal carcinoma, endocrine cancers, endometrial cancer, ependymoma, esophageal cancer, Ewing's sarcoma, extra-hepatic bile duct cancer, eye cancer, eye: melanoma, retinoblastoma, fallopian tube cancer, fanconi anemia, fibrosarcoma, gall bladder cancer, gastric cancer, gastrointestinal cancers, gastrointestinal carcinoid tumor, genitourinary cancers, germ cell tumors, gestational-trophoblastic disease, glioma, gynecological cancers, hematological malignancies, hairy cell leukemia, head and neck cancer, hepatocellular cancer, hereditary breast cancer, histiocytosis, Hodgkin's disease, human papillomavirus, hydatidiform mole, hypercalcemia, hypopharynx cancer, intraocular melanoma, islet cell cancer, Kaposi's sarcoma, kidney cancer, Langerhan's cell histiocytosis, laryngeal cancer, leiomyosarcoma, leukemia, L1-Fraumeni syndrome, lip cancer, liposarcoma, liver cancer, lung cancer, lymphedema, lymphoma, Hodgkin's lymphoma, non-Hodgkin's lymphoma, male breast cancer, malignant-rhabdoid tumor of kidney, medulloblastoma, melanoma, Merkel cell cancer, mesothelioma, metastatic cancer, mouth cancer, multiple endocrine neoplasia, mycosis fungoides, myelodysplastic syndromes, myeloma, myeloproliferative disorders, nasal cancer, nasopharyngeal cancer, nephroblastoma, neuroblastoma, neurofibromatosis, Nijmegen breakage syndrome, non-melanoma skin cancer, non-small cell lung cancer (NSCLC), ocular cancers, esophageal cancer, oral cavity cancer, oropharynx cancer, osteosarcoma, ostomy ovarian cancer, pancreas cancer, paranasal cancer, parathyroid cancer, parotid gland cancer, penile cancer, peripheral-neuroectodermal tumors, pituitary cancer, polycythemia vera, prostate cancer, rare-cancers-and-associated-disorders, renal cell carcinoma, retinoblastoma, rhabdomyosarcoma, Rothmund thomson syndrome, salivary gland cancer, sarcoma, schwannoma, Sezary syndrome, skin cancer, small cell lung cancer (SCLC), small intestine cancer, soft tissue sarcoma, spinal cord tumors, squamous-cell-carcinoma-(skin), stomach cancer, synovial sarcoma, testicular cancer, thymus cancer, thyroid cancer, transitional-cell-cancer-(bladder), transitional-cell-cancer-(renal-pelvis-/-ureter), trophoblastic cancer, urethral cancer, urinary system cancer, uroplakins, uterine sarcoma, uterus cancer, vaginal cancer, vulva cancer, Waldenstrom's-macroglobulinemia, Wilms' tumor.

In other embodiments, the composition of the invention could also be used for generating large numbers of CD8⁺ or CD4⁺ CTL, for adoptive transfer to immunodeficient individuals who are unable to mount normal immune responses. For example, antigen-specific CD8⁺ CTL can be adoptively transferred for therapeutic purposes in individuals afflicted with HIV infection (Koup et al., 1991, J. Exp. Med. 174: 1593-1600; Carmichael et al., 1993, J. Exp. Med. 177: 249-256; and Johnson et al., 1992, J. Exp. Med. 175: 961-971), malaria (Hill et al., 1992, Nature 360: 434-439) and malignant tumours such as melanoma (Van der Brogen et al., 1991, Science 254: 1643-1647; and Young and Steinman 1990, J. Exp. Med., 171: 1315-1332).

In still other embodiments, the composition is suitable for treatment or prophylaxis of a viral, bacterial or parasitic infection. Viral infections contemplated by the present invention include, but are not restricted to, infections caused by HIV, Hepatitis, Influenza, Japanese encephalitis virus, Epstein-Barr virus and respiratory syncytial virus. Bacterial infections include, but are not restricted to, those caused by Neisseria species, Meningococcal species, Haemophilus species Salmonella species, Streptococcal species, Legionella species and Mycobacterium species. Parasitic infections encompassed by the invention include, but are not restricted to, those caused by Plasmodium species, Schistosoma species, Leishmania species, Trypanosoma species, Toxoplasma species and Giardia species.

The effectiveness of the immunization may be assessed using any suitable technique. For example, CTL lysis assays may be employed using stimulated splenocytes or peripheral blood mononuclear cells (PBMC) on peptide coated or recombinant virus infected cells using ⁵¹Cr or Alamar Blue™ labeled target cells. Such assays can be performed using for example primate, mouse or human cells (Allen et al., 2000, J. Immunol. 164(9): 4968-4978 also Woodberry et al., infra). Alternatively, the efficacy of the immunization may be monitored using one or more techniques including, but not limited to, HLA class I tetramer staining—of both fresh and stimulated PBMCs (see for example Allen et al., supra), proliferation assays (Allen et al., supra), ELISPOT assays and intracellular IFN-γ staining (Allen et al., supra), ELISA Assays—for linear B cell responses; and Western blots of cell sample expressing the synthetic polynucleotides.

In some embodiments, the composition comprises a nucleic acid construct from which an antigen that corresponds to the target antigen is expressible. Administration of such constructs to a mammal, especially a human, may include delivery via direct oral intake, systemic injection, or delivery to selected tissue(s) or cells. Delivery of the constructs to cells or tissues of the mammal may be facilitated by microprojectile bombardment, liposome mediated transfection (e.g., lipofectin or lipofectamine), electroporation, calcium phosphate or DEAE-dextran-mediated transfection, for example. A discussion of suitable delivery methods may be found in Chapter 9 of Ausubel et al., (1994-1998, supra).

The step of introducing the expression vector into the selected target cell or tissue will differ depending on the intended use and species, and can involve one or more of non-viral and viral vectors, cationic liposomes, retroviruses, and adenoviruses such as, for example, described in Mulligan, R. C., (1993 Science 260 926-932). Such methods include, for example:

A. Local application of the expression vector by injection (Wolff et al., 1990, Science 247 1465-1468), surgical implantation, instillation or any other means. This method can also be used in combination with local application by injection, surgical implantation, instillation or any other means, of cells responsive to the protein encoded by the expression vector so as to increase the effectiveness of that treatment. This method can also be used in combination with local application by injection, surgical implantation, instillation or any other means, of another factor or factors required for the activity of the protein.

B. General systemic delivery by injection of DNA, (Calabretta et al., 1993, Cancer Treat. Rev. 19 169-179), or RNA, alone or in combination with liposomes (Zhu et al., 1993, Science 261 209-212), viral capsids or nanoparticles (Bertling et al., 1991, Biotech. Appl. Biochem. 13 390-405) or any other mediator of delivery. Improved targeting might be achieved by linking the polynucleotide/expression vector to a targeting molecule (the so-called “magic bullet” approach employing, for example, an antigen-binding molecule), or by local application by injection, surgical implantation or any other means, of another factor or factors required for the activity of the protein encoded by the expression vector, or of cells responsive to the protein. For example, in the case of a liposome containing antisense IL-10 polynucleotides, the liposome may be targeted to skin cancer cells, e.g., squamous carcinoma cells, by the incorporation of immuno-interactive agents into the liposome coat which are specific the EGF receptor, which is expressed at higher levels in skin cancer.

C. Injection or implantation or delivery by any means, of cells that have been modified ex vivo by transfection (for example, in the presence of calcium phosphate: Chen et al., 1987, Mole. Cell Biochem. 7 2745-2752, or of cationic lipids and polyamines: Rose et al., 1991, BioTech. 10 520-525), infection, injection, electroporation (Shigekawa et al., 1988, BioTech. 6 742-751) or any other way so as to increase the expression of the polynucleotide in those cells. The modification can be mediated by plasmid, bacteriophage, cosmid, viral (such as adenoviral or retroviral; Mulligan, 1993, Science 260 926-932; Miller, 1992, Nature 357 455-460; Salmons et al., 1993, Hum. Gen. Ther. 4 129-141) or other vectors, or other agents of modification such as liposomes (Zhu et al., 1993, Science 261 209-212), viral capsids or nanoparticles (Bertling et al., 1991, Biotech. Appl. Biochem. 13 390-405), or any other mediator of modification. The use of cells as a delivery vehicle for genes or gene products has been described by Barr et al., 1991, Science 254 1507-1512 and by Dhawan et al., 1991, Science 254 1509-1512. Treated cells can be delivered in combination with any nutrient, growth factor, matrix or other agent that will promote their survival in the treated subject.

In order that the invention may be readily understood and put into practical effect, particular preferred embodiments will now be described by way of the following non-limiting examples.

### EXAMPLES

### Example 1

### Overcoming Original Antigenic Sin to Generate New CD8 T Cell IFN-Gamma Responses in an Antigen Experienced Host

### Materials and Methods

### Mice

4-8 weeks old adult female C57BL/6 (H-2b) mice were purchased specific pathogen free (SPF) from the Animal Resource Centre (ARC, Perth, Australia), Human papillomavirus 16 E7 (RAHYNIVTF) MHC class I restricted T cell receptor beta chain transgenic mice on a C57BL/6J background were produced in the lab as described elsewhere (Matsumoto, 2004, J Natl Cancer Inst 96:1611-1619). Mice were kept under SPF conditions throughout, and all experiments were approved by and performed in compliance with the guidelines of the University of Queensland animal experimentation ethics committee.

### Cell Lines and Peptides and Antibodies

Spodoptera frugiperda (Sf-9) cells (Life Technique) were maintained in Sf-900 II medium with Sf-9 II Supplement (Life Technique) and 10% fetal bovine serum (FBS) (CSL, Melbourne) at 27° C. Anti-IL-10R hybridoma (3B1.3a) was kindly provided by Dr. Warwick Britton of Centenary Institute, University of Sydney and was maintained in RPMI-1640 (Invitrogen, USA) with 10% FBS.

For production of anti-IL-10R MAb, hybridoma cells were cultured in RPMI with 1% FBS for 72 hours, supernatants were collected and passed through a Protein G column (Sigma-Aldrich), and eluted by running 100 mM glycine (Sigma-Aldrich) through the column. Eluted antibodies were dialyzed extensively against phosphate buffered saline (PBS) (0.15M NaCl; 0.02M PO4; pH 7.4) and the concentration of antibody was measured as previously described (Liu et al., 2003, J Immunol 171:4765-4772).

Anti-CD4 MAb (GK1.5) and anti-CD8 MAb (2.43) were produced from ascites. Anti CD4-FITC MAb (RM4-4), FITC rat IgG2a (R35-95), anti-IL-10 MAb (JES5-16E3), anti-Gr-1 MAb (RB6-85C); anti-CD45R/B220 MAb (RA3-6B2); and anti-CD16/CD32 MAb FcγII/II (2.4G2) were purchased from BD PharMingen (San Diego, Calif.)

The MHC Class I (H-2 D^{b}) restricted HPV16 E7 peptide RAHYNIVTF was synthesised and purified by Chiron Mimotopes (Melbourne, Australia). Aluminium hydroxide gel was purchased from Superfos Biosector (Vedbaek, Denmark), and incomplete Freunds adjuvant from Sigma-Aldrich (St. Louis, Mo.).

### Isolation of Mouse Mononuclear Cells and Flow Cytometric Analysis

Isolation of mouse blood mononuclear cells was performed by density gradient centrifugation. Briefly, 200 μL of venous blood was added to PBS containing 0.2% EDTA Na₂ (Sigma-Aldrich) and overlayed to 1 ml of Histopaque (Sigma-Aldrich). After centrifugation at 400 g for 15 minutes at 22 C, the interlayer was washed extensively with PBS containing 0.1% bovine serum albumin and 0.1% NaN3, then exposed to FITC- conjugated MAb at room temperature for 15 minutes, and analysed using a Becton Dickinson FACSCaliber Flow cytometer and Cellquest (Becton Dickinson) software.

### Production of Recombinant VLPs

Construction of baculovirus encoding Human papillomavirus 6bL1, BPV1L1 VLP (L1VLP) and BPV1L1/L1PV16E7CTL (L1E7VLPs) recombinant protein has been described (Peng et al., 1998 Virology 240:147-157). VLPs were purified from the nuclei of SF9 cells infected with L1 recombinant baculovirus by CsCl gradient centrifugation as previously described (Liu et al., 2003, supra). Samples were subjected to analysis by transmission electron microscopy and immunoblotting to confirm the identity and integrity of the VLPs. For immunoblot analysis, protein samples were diluted in SDS-PAGE sample buffer, electrophoresed through a 10% SDS-PAGE gel, and transferred to nitrocellulose membrane. The membrane was probed with the anti-L1 monoclonal antibody MC15 (Kulski et al., 1998, Virology 243:275-282). Bound antibody was detected by incubation of the membrane with horseradish peroxidase conjugated sheep anti-mouse antibody (Silenus) and visualized using enhanced chemiluminescence (Amersham). For electron microscopy, CsCl gradient-purified and dialyzed VLP samples were mounted onto carbon-coated grids, stained with 2% ammonium molybdate, pH 6.2, and examined with a Hitachi H-800 electron microscope.

### Immunization of Mice

Groups of three or five mice were immunized as indicated with 30 or 50 μg of VLPs with or without aluminium hydroxide gel (“alum”). Mice were lightly anaesthetized with Isofluorane (Abbott) during immunization. VLPs were in 50 μL of PBS or mixed with equal volume of alum. For in vivo neutralizing experiments, 0.5-1 mg of monoclonal antibodies or of normal rat serum was administered intraperitoneally.

### ELISA for IL-10 IL-5 and IFN-γ Cytokines from Culture Supernatants

ELISA for IL-5, IL-10 and IFN-γ (R&D system, USA) was performed as described previously, according to the manufacturer's recommended procedures (Liu et al., 2003, supra).

### ELISPOT

ELISPOT was performed as described (Khammanivong et al., 2003, Immunol Cell Biol 81:1-7). Briefly, single spleen cell or lymph node suspensions were added to membrane base 96 well plates (Millipore) coated with anti-IFN γ (BD PharMingen) with or without added IL-2 (Life Techniques). Peptide was added at various concentrations and cells held at 37° C. with peptide for 18 hours. Antigen specific IFN γ secreting cells were detected by sequential exposure of the plate to biotinylated anti-IFN gamma (BD PharMingen), avidin—horseradish peroxidase (Sigma-Aldrich) and DAB (Sigma-Aldrich).

### Positive Selection of Mouse Spleen CD11c+ Cells

C57 BL/6J mouse spleens were held in 1 mg/ml of Collagenase D (Roche) and 500 ul of Collagenase D was injected into each spleen. The spleens were then cut in smaller pieces, held in 5 ml of Collagenase D for 45-60 minutes at 37° C., and passed through a steel mesh. The cells were counted, washed in RPMI with 2% FBS, and resuspended in 400 mL RPMI with 2% FBS per 108 total cells. 100 μL of MACS CD11c Microbeads (Miltenyi Biotec) were added, and held for 15 min at 6-12° C. After washing, cells were resuspended in 500 μl per 10⁸ cells. CD11c positive cells were positively selected with a LS column (Miltenyi Biotec) according to the manufacturer's protocols. The purity of CD11c+ cells was around 80% as assessed by flow cytometry.

### Selection of CD4 Cells

To generate a lymphocyte population enriched for antigen primed CD4⁺ T cells, mice were immunized with L1VLPs twice, and draining inguinal lymph nodes were removed 7 days after the second immunization. Cells were passed through a 70 μm nylon membrane (BD, PharMingen) and re-suspended in 1 ml RPMI+2% FBS. For every 10⁸ cells: Anti-Gr-1 MAb 8 μL, Anti-B220 MAb 6 μL, Anti-MHC II (1-A^{b}) MAb 5 μL, Anti-FcγII/III MAb 4 μL, and anti-CD8 MAb 8 μL were added. After incubation at room temperature for 15 minutes, the cells were washed and re-suspended in 1 ml of RPMI+2% FBS. Anti-rat-MACS beads (Miltenyi Biotec) were added at 100 μL/10⁸ cells for 15 minutes at 9° C., washed with RPMI media and passed through an LS column following the manufacturer's protocols. For positive selection of CD4⁺ T cells, cells were held with anti-CD4 MAb (RM4-4) for 1 hour at 4° C., and then with Rat anti-CD4 Dynabead beads for 1 hour at 4° C. on a rotating mixer. CD4⁺ cells were then positively selected using a Magnetic Particle Concentrator (Dynal Biotech) following the manufacturers instructions.

### In Vitro Activation of E7 TCR Transgenic CD8 T Cells by APC Exposed to Chimeric E7 VLPs

10⁵ CD11+ cells were exposed to 40 μg of BPVL1 VLPs, BPV1-HPV16E7 chimeric VLPs or HPV6 VLPs for 18 hours at 37° C. After extensive washing, cells were placed in U shape 96-well tissue culture plates (Cellstar), and different numbers of splenocytes, depleted of adherent cells by exposure to plastic at 37° C. for 2 hours, from C57B1/6 mice, or from C57B1/6 mice with a transgene TCR β-chain specific for the MHC Class I restricted E7 peptide RAHYNIVTF, designated E7 T cells, were added. Approximately 50% of T cells from the TCR β-chain transgenic C57B1/6 animals bind E7 peptide tetramers and secrete IFN-γ in response to E7 peptide pulsed targets (Matsumoto et al., J Natl Cancer Inst. 96 (21):1611-1619, 2004). Cells were held for 2 days at 37° C., and supernatants collected for cytokine measurement. ³H thymidine was added to the culture plate for another 16 hours and T cell proliferation assayed as described (Fernando et al., 1998, J Immunol 161:2421-2427). In some experiments, CD4 enriched lymphocytes from mice immunized with L1VLPs or unrelated antigen were added for 18 hours to CD11c+ cells exposed to VLPs. E7 T cells and 15 μg/ml of GK1.5 blocking antibody were then added and cells cultured and cytokine secretion and proliferation assessed after 48 hours as described above.

### Statistical Analysis

Statistical analysis was performed using the two tailed Student's t test.

### Results and Discussion

### Carrier Primed CD4+ Cells Mediate Inhibition of IFN-γ Secretion by CD8 T Cells In Vivo

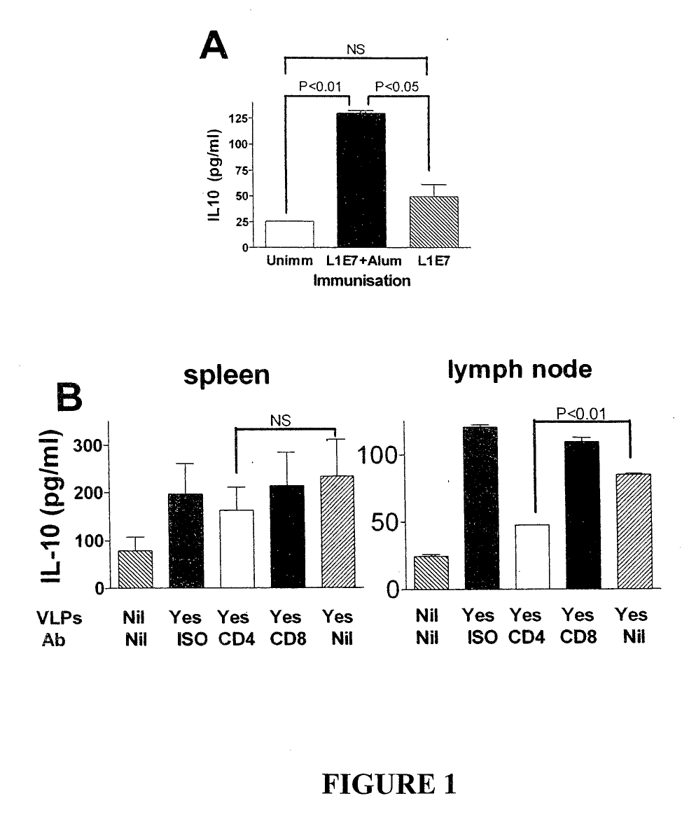

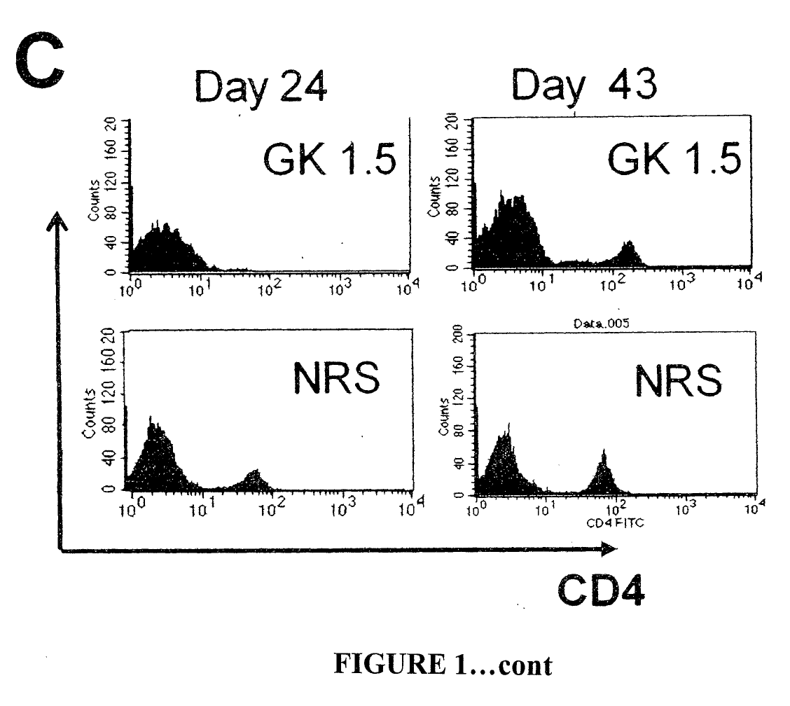

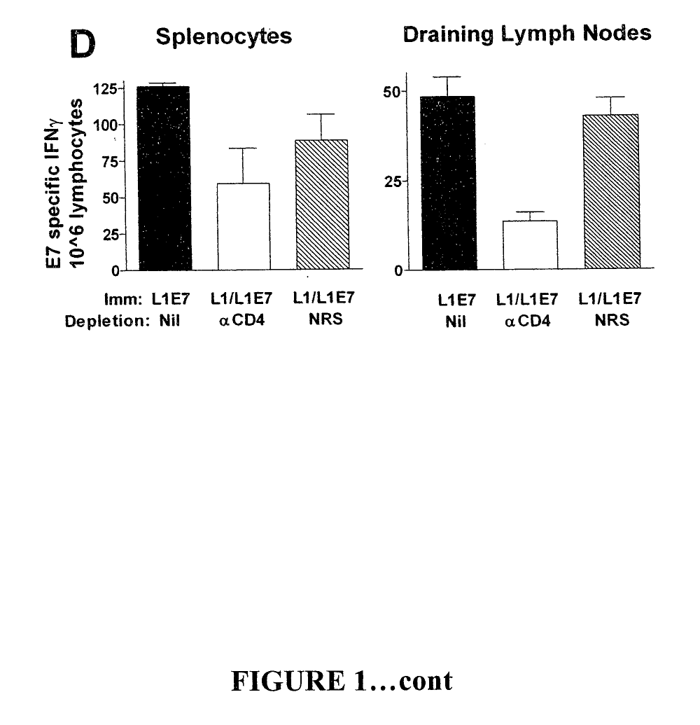

Administration of a MHC class I restricted epitope coupled to a virus capsid carrier induces epitope specific IFN-γ secreting CD8 T cells (Peng et al., 1998, supra; Liu et al., 2000, Virology 273:374-382). However, activation of an IFN-γ response to a new epitope associated with a virus capsid is inhibited by pre-existing immunity to the carrier virus capsid, an observation referred to as original antigenic sin (Liu et al., 2003, supra; Da Silva, 2001 supra). The present inventors have previously demonstrated that such inhibition is localized to the site of carrier antigen priming, and requires IL-10 production (Liu et al., 2003, supra). In this study, they define the mechanism of the observed inhibition and a means to overcome it. Since IL-10 secreting T cells with regulatory function are mainly CD4+ (Sakaguchi, 2004, Annu Rev Immunol 22:531-562), it was decided to establish whether immune CD4 cells were necessary in vivo for the inhibition of IFN-γ responses that had been observed previously. The present inventors, therefore, depleted CD4+ cells from BPV1 L1 virus capsid primed animals that were unable to mount an IFN-γ response to a new E7 epitope associated with the virus capsid. They then observed recovery of a naïve CD4 population over 3 weeks (FIG. 1C), and tested for recovery of the ability to mount an IFN-γ response to the E7 epitope by immunization with L1E7 VLPs. As expected, E7 epitope specific IFN γ responses were induced by L1E7 VLPs in a naïve animal, and were not induced by L1E7 VLPs in L1 VLP primed, non-depleted mice. However, E7 specific IFN-γ secreting T cells were readily detected in L1 VLP immunized mice that were depleted of antigen experience CD4 cells and allowed to recover a naïve CD4 population before immunization with L1E7 VLPs (FIG. 1D). These results confirm the inventors' previous findings, and demonstrate in vivo that viral capsid specific CD4⁺ T cells are required for inhibition of the epitope specific IFN-γ response.

As inhibition of induction of E7 specific T cell IFN-γ responses by L1E7 VLPs in L1VLP primed mice is dependent on IL-10 and also L1VLP primed CD4⁺ T cells, it was proposed that IL-10 would be produced by primed CD4⁺ cells in the lymph nodes draining the L1VLP injection site. To test this, mice were immunized twice with L1E7 VLP and cytokine secretion by cells from the draining lymph node examined. L1E7VLPs injected without adjuvant, or with alum adjuvant, enhanced antigen specific IL-10 production by draining lymph node cells stimulated in vitro with L1E7VLPs (FIG. 1A). IL-10 production was significantly reduced by anti-CD4 treatment of lymph node cells, but not by anti-CD8 treatment (FIG. 1B). L1E7VLP immunization similarly enhanced IL-10 production in response to L1E7VLP antigen by splenocytes from immunized animals. Thus, immunization with VLPs induces IL-10 secretion in draining lymph nodes by VLP specific CD4 positive T cells, suggesting that these VLP specific CD4 cells contribute to the IL-10 dependent suppression of induction, in response to immunization with a new MHC Class I restricted epitope, of IFN-γ secreting T cells.

### Viral Capsid Primed CD4⁺ Cells Inhibit Antigen Specific IFN γ Secretion In Vitro

E7 peptide specific T cell responses are observed in L1VLP primed hosts immunized with E7 protein and L1VLPs, if the E7 peptide is not covalently linked to the VLPs (Liu et al., 2003, supra). This suggests the hypothesis that if the same DC present L1VLP peptide to CD4 T cells and E7 peptide to CD8 T cells the CD4 cells, by secreting IL-10, locally influence the fate of E7 specific CD8 cells. To investigate this hypothesis, the present inventors set up an in vitro system in which CD11c⁺ cells isolated from mouse spleen (called DC hereafter) were exposed to L1 VLPs, L1E7 VLPs or unrelated HPV6L1 VLPs for 18 hours. Activation in vitro of naïve E7 specific (TCR transgenic) CD8 T cells by the antigen pulsed DC was then assessed as IFN-γ secretion and T cell proliferation. Activation in vitro of E7 specific CD8⁺ T cells was observed for DC exposed to L1E7 VLPs but not L1 VLP or HPV6L1 VLP, as expected (FIG. 2A). No significant IFN-γ secretion was observed from CD8⁺ or CD4⁺ T cells exposed to unpulsed DC or DC exposed to L1VLPs (FIG. 2B, C), indicating the specificity of the in vitro system.

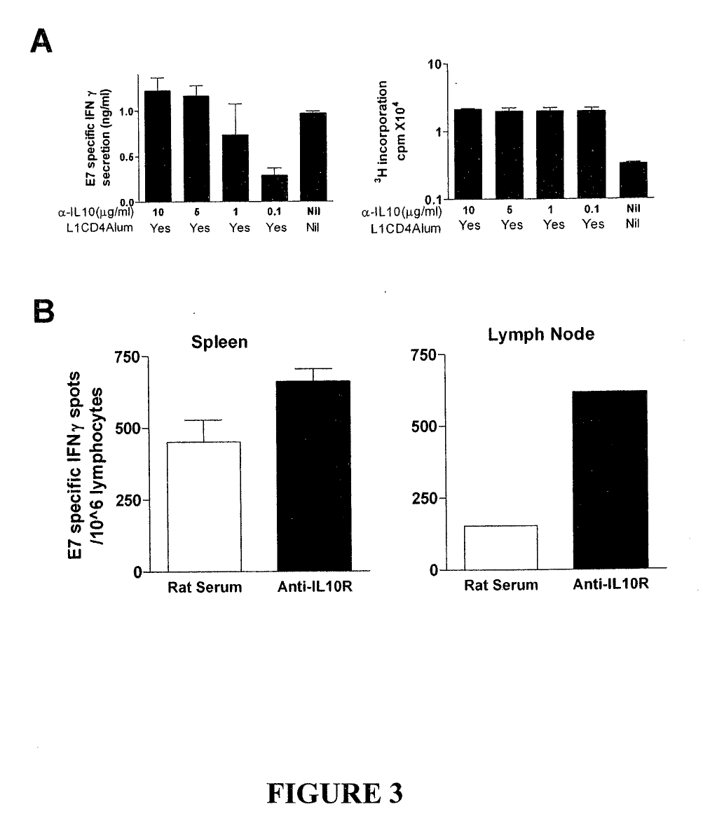

To examine the effect of CD4 cells from animals immunized with L1VLP on CD8 T cell activation in this in vitro system, the present inventors added CD4⁺ T cells from draining lymph nodes of L1VLP immunized mice, or unimmunized mice. CD4⁺ T cells were co-cultured with previously VLP exposed DCs for 18 hours, before addition of E7 TCR transgenic T cells. Surprisingly, E7 specific CD8⁺ T cell proliferation and IFN-γ secretion were increased when CD4 cells from L1 VLP immunized mice were added (FIG. 2B), though, as expected, CD4 T cells from naïve mice did not influence the E7 specific IFN y secretion by CD8 T cells. These data suggested that the majority of the L1 VLP specific CD4 cells from L1VLP immunized mice, upon in vitro exposure to antigen, were functioning as helper T cells, and that local in vivo environment and intact lymph node structure might be important for CD4⁺ induced suppression of CD8⁺ priming by antigen pulsed DC. As CD4⁺ T cells from mice immunized with VLPs secreted more IL-10 if the VLPs were co-administered with alum, the present inventors tested whether VLP and alum primed CD4⁺ T cells could inhibit activation of E7 specific T cells by L1E7 VLP exposed DC. CD4 cells from animals immunized with alum and VLP, in contrast to CD4⁺ cells from animals immunized with VLP alone, significantly inhibited activation of E7 specific T cell IFN-γ secretion (FIG. 2C). To investigate whether this inhibition was mediated by IL-10 as predicted from the inventors' in vivo observation, they first assayed supernatants from different CD4⁺ cell and DC cultures for IL-10. As expected, IL-10 secretion was significantly higher from cultures including CD4⁺ cells from VLP immunized animals than from cultures with CD4⁺ cells from control immunized animals (FIG. 2D). Further, neutralization of IL-10 in vitro restored the activation by L1E7 VLP primed DC of IFN-γ secretion by E7 peptide specific T cells (FIG. 3A). Thus, viral capsid primed CD4⁺ cells secrete IL-10 upon interaction with DCs presenting L1E7. Interaction between CD4⁺ cells and DC prevents the subsequent activation by DC of IFN-γ secretion from E7 specific CD8 restricted T cells, and inhibition of IFN-γ secretion is dependent on IL-10.

### DCs Exposed to VLP Specific CD4 T Cells Prime Naïve CD8 T Cells to Secrete IL-5

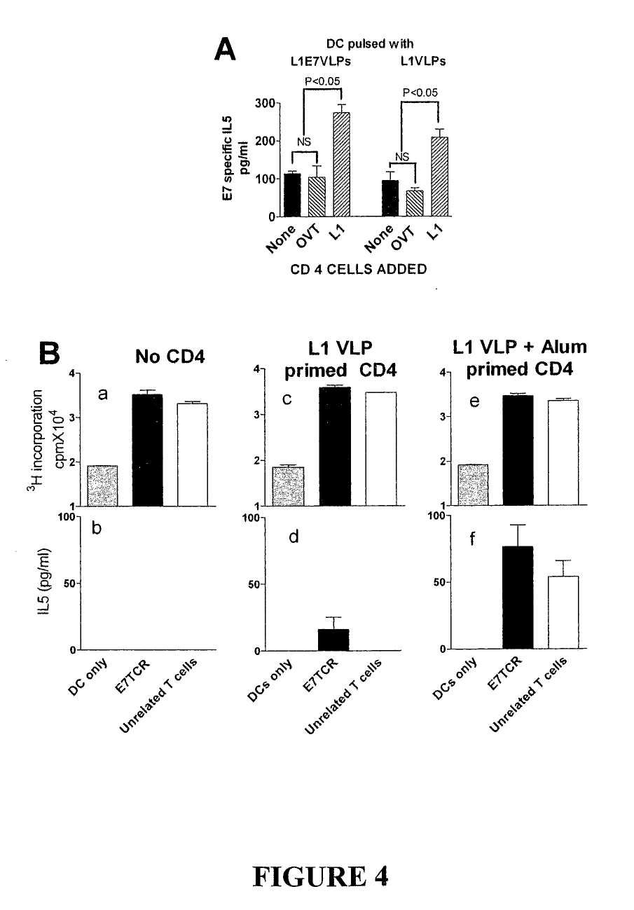

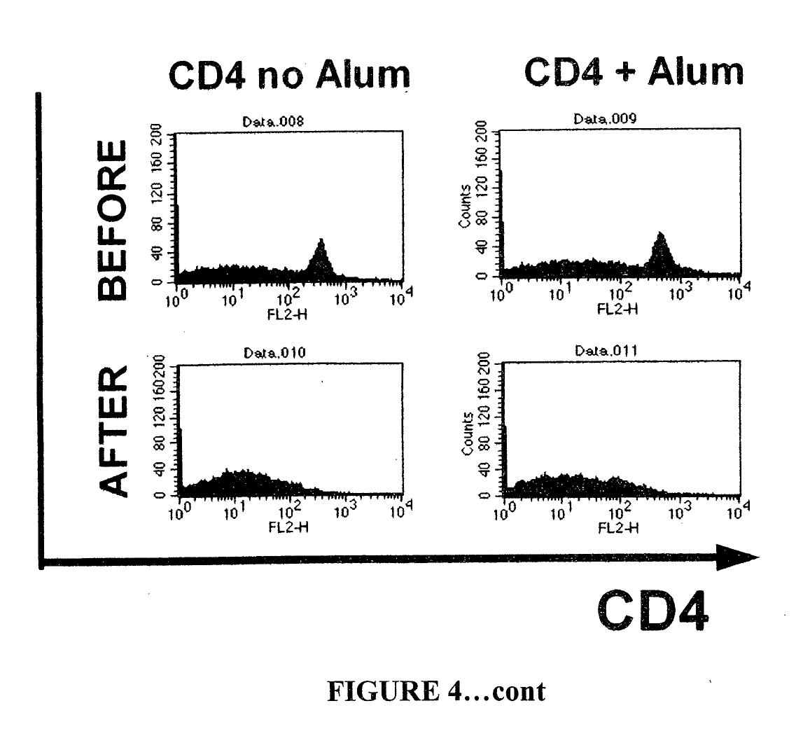

Although IFN γ secretion by E7 specific CD8⁺ T cells was inhibited when immune CD4 cells were co-cultured with DCs presenting L1E7, T cell proliferation was increased (FIG. 2C), suggesting that activation by antigen exposed DC of E7 TCR transgenic T cells might result in different functional outcomes according to the cytokine environment in which antigen presentation occurs. To investigate whether the observed suppression by IL-10 secreting CD4⁺ T cells of IFN-γ production by CD8⁺ T cells represented a failure to develop an effector phenotype, or alternatively a shift in effector phenotype from Tc1 to Tc2 type, the present inventors measured IL-5 production by cells in co-culture. IL-5 secretion by CD8⁺ T cells was higher upon addition of CD4 T cells from animals immunized with L1 VLPs and alum, than on addition of unrelated CD4⁺ T cells (FIG. 4A). To clarify whether E7 specific CD8⁺ T cells were the source of the enhanced IL-5 secretion, CD4 cells were removed after 18 hours co-culture with the antigen exposed DC (FIG. 4, FACS result), and the “educated” DCs were then cultured with E7TCR CD8 T cells, or with unrelated CD8⁺ T cells. E7 specific T cell proliferation was similar whether the DC had been educated by L1 specific CD4 cells, or had not been exposed to CD4⁺ T cells (FIG. 4B a, c, e). However, significantly more IL-5 was produced by E7 specific T cells culture with DC that had been educated by antigen primed CD4⁺ cells, particularly if the primed CD4⁺ cells were induced with alum to secrete large amounts of IL-10 (FIG. 4B b, d, f). Of interest, high levels of IL-5 secretion were also observed from unrelated naïve T cells (FIG. 4B,f) exposed to DC that had been educated by IL-10 secreting CD4⁺ T cells, suggesting that such DC could induce CD8⁺ T cells non specifically to secrete IL-5. Thus, the cytokines produced by CD8 cells exposed to antigen primed DC in vitro can be determined by the prior education of the DC by CD4⁺ T cells recognizing their cognate antigen presented by the DC.

### Restoration of a CD8⁺ CTL Response Through Neutralization of IL-10

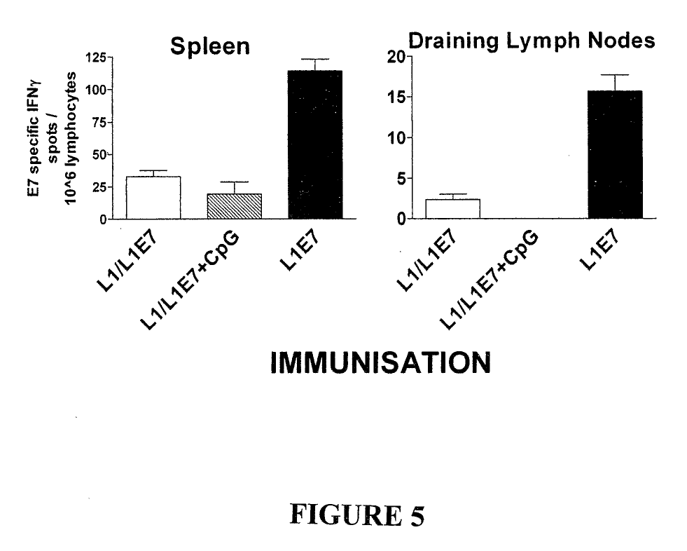

The inventors tested methods that might overcome the inhibition of a Tc1 type CD8 response to epitopes linked to a carrier protein, consequent upon prior priming of the animal to the carrier protein. As patients with cancer or chronic viral infection commonly have ineffective immune responses to virus and tumor specific antigens, characterized by antibody and lack of tumor or virus antigen specific CTL effectors, overcoming such inhibition may be important for immunotherapy. Administration of VLPs together with CpG DNA as a stimulus to the innate immune system can increase the magnitude of the induced CD8⁺ immune response (Storni, 2002, J Immunol 168:2880-2886), so the present inventors immunized viral capsid protein primed mice with L1E7 with CpG, but saw no induction of E7 specific CD8 IFN-γ responses (FIG. 5A,B). Thus increasing the immunogenicity of antigen in a primed host through better activation of DC could not overcome the observed inhibition of new CD8⁺ Tc1 responses by prior priming. The inventors then tested in vitro and in vivo whether neutralizing IL-10 could restore to an L1 primed animal the ability to mount an E7 specific IFN-7 response upon immunization with L1E7. Different concentrations of neutralizing anti-IL-10 antibody were added to the in vitro DC/CD4/CD8 co-culture system, and E7 specific CD8⁺ T cell IFN-γ secretion was restored by anti IL-10 in a dose dependent manner, while T cell proliferation was not altered (FIG. 3A). These results confirmed the inventors' finding that E7TCR activated in the presence of IL-10 are capable of responding by proliferation but fail to produce IFNγ. Next, they investigated whether neutralizing the effect of IL-10 in vivo by blocking IL10 receptor, through administration of anti-IL10 receptor antibody could restore the E7 specific IFN-γ response to L1E7 in L1VLPs primed mice. Mice primed with L1VLPs were administered a blocking antibody to the IL-10 receptor, or rat serum, at the time of immunization with L1E7 VLPs. IFN-γ secreting E7 specific CD8 T cells were observed in mice receiving anti-IL-10R but not in mice receiving normal rat serum, showing that temporary neutralization of IL-10 could permit induction of E7 specific IFN-γ secreting CD8 T cells by L1E7 despite prior priming to L1 (FIG. 3B).

### Example 2

### Generation of a New Proinflammatory Response Through Neutralization of IL-10

### Materials and Methods

### Immunization of Mice

Mice were immunized with 50 μg of HPV16E7 and 10 μg of QuilA. Some mice also received an IL-10 inhibitor in the form of 0.3 mg of anti-IL-10 receptor antibody, intraperitoneally.

### Skin Graft

Whole ears from donor K14E7 mice, where HPV16E7 is only expressed in epithelial cells, were surgically removed, and dorsal and ventral surfaces were separated. Transgenic graft were placed on the flanks of C57BL/6 mice, held in place with antibiotic permeated Vaseline gauze (Bactigrass, Smith and Nephew, London), covered with micro-pore tape and elastic bandages (CoFlex; Andover, Salisbury, Mass.) for 7 days, and accessed as technically successful if they were adherent and vascularized on day 7. Skin grafts were observed at 3 times weekly for the duration of study and the data in Table 1 summarize the graft condition on day 14 after grafting.

### Results and Discussion

The inventors have previously determined that skin grafts expressing E7 from a keratin 14 (K14) promoter are not rejected by naïve syngeneic animals, and show no evidence of an inflammatory response or of inducing measurable systemic immunity to E7. Immunization of animals bearing such grafts induces immunity to E7 measurable as antibody, delayed type hypersensitivity (DTH), and E7 specific CTL but has no effect on the E7 expressing grafts (Matsumoto, K., et al., 2004, J Natl Cancer Inst 96:1611; Dunn, L. A., Met al., 1997. Virology 235:94). The grafts are however susceptible to rejection by E7 specific CTL, as rejection can be achieved by passive transfer of 10⁶ E7 TCR transgenic T cells plus E7 immunization.

The inventors exploited this skin graft model to demonstrate whether coadministration of an E7 vaccine and IL-10 inhibition enhances the immune response to the E7 protein antigen of HPV 16. In particular, they immunized animals bearing K14E7 grafts with E7 (as HPV16L1 E7 VLPs), optionally together with anti-IL-10 receptor antibody, and observed the inflammatory response induced by such immunizations. The results presented in Table 1 show that coadministration of IL-10 inhibitor markedly enhances the local inflammatory response in the E7 bearing graft. Specifically, 2 of 8 mice so treated developed either partial graft rejection (2 animals) and severe inflammation in the grafts whereas without administration of IL-10 inhibitor no graft rejection and only rare cases of inflammation (<1 animal in 10) were observed.

TABLE 1

| | GROUP 1 | GROUP 2 |
|---|---|---|
| No of recipients | 4 | 8 |
| Graft | K14E7 | K14E7 |
| 50 μg of E7gst | | |
| 10 μg of QuilA | Yes | Yes |
| 0.3 mg of anti-IL-10R antibody | No | Yes |
| Partial rejection of graft on day 14 | 0 | 2 |

The above results demonstrate that E7 immunization plus IL-10 neutralization generates stronger E7 specific immune responses, than those immunized with E7 alone. Also, as rejection in this model is dependent on E7 specific CTL, these results also demonstrate that neutralization of IL-10 at the time of administration of an E7 vaccine generates an enhanced E7 specific CTL response.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

What is claimed is:

1. A composition for stimulating an immune response against a target antigen in a subject that is naïve to the target antigen or that has previously raised an immune response to the target antigen, the composition comprising an immune stimulator that stimulates or otherwise enhances an immune response to the target antigen and an inhibitor of IL-10 function.

2. A composition according to claim 1, wherein the immune stimulator is selected from an antigen that corresponds to at least a portion of the target antigen, an antigen-binding molecule that is immuno-interactive with the target antigen and an immune-stimulating cell that stimulates or otherwise enhances an immune response to the target antigen.

3. A composition according to claim 1, wherein the target antigen is associated with a disease or condition of interest.

4. A composition according to claim 1, wherein the target antigen is produced by a pathogenic organism or a cancer.

5. A composition according to claim 2, wherein the antigen that corresponds to at least a portion of the target antigen is in soluble form.

6. A composition according to claim 2, wherein the antigen that corresponds to at least a portion of the target antigen is a particle or cell.

7. A composition according to claim 2, wherein the antigen that corresponds to at least a portion of the target antigen is presented by an antigen-presenting cell.

8. A composition according to claim 2, wherein the immune-modulating agent is an antigen-binding molecule, which binds to or otherwise interacts with the target antigen so as to reduce its level or functional activity.

9. A composition according to claim 2, wherein the immune-stimulating cell is an immune effector cell.

10. A composition according to claim 9, wherein the immune effector cell is selected from antigen-specific T lymphocytes.

11. A composition according to claim 1, further comprising at least one other immune stimulator, which stimulates or otherwise enhances an immune response to the target antigen or to a plurality of target antigens.

12. A composition according to claim 1, wherein the subject has previously raised an immune response to the target antigen and the immune stimulator comprises an antigen that corresponds to the target antigen, wherein the amino acid sequence of the corresponding antigen is the same as the amino acid sequence of the at least said portion.

13. A composition according to claim 1, wherein the subject has previously raised an immune response to the target antigen and the immune stimulator comprises an antigen that corresponds to the target antigen, wherein the amino acid sequence of the corresponding antigen is distinguished from the amino acid sequence of the at least said portion by the addition, deletion or substitution of at least one amino acid residue.

14. A composition according to claim 12 or claim 13, wherein the corresponding antigen is a naturally-occurring antigen to which the subject has already raised an immune response.

15. A composition according to claim 1, wherein the inhibitor of IL-10 function is selected from soluble or defective IL-10 receptors or fragments thereof, cells expressing IL-10 receptors or fragments thereof, antigen-binding molecules that are immuno-interactive with IL-10 or an IL-10 receptor, nucleic acids that inhibit the expression of an IL-10 gene or the functional activity of an IL-10 expression product or small molecule inhibitors of IL-10.

16. A composition according to claim 1, further comprising a pharmaceutically acceptable carrier or diluent.

17. A composition according to claim 1, further comprising an adjuvant that enhances the effectiveness of the immune stimulation.

18. A composition according to claim 17, wherein the adjuvant delivers the antigen to the class I major histocompatibility pathway.

19. A composition according to claim 17, wherein the adjuvants is selected from a saponin-containing compound and a cytolysin that mediates delivery of antigens to the cytosol of a target cell.

20. A composition according to claim 19, wherein the cytolysin is linked to, or otherwise associated with, the antigen.

21. A composition according to claim 19, wherein the cytolysin mediates transfer of the antigens from the vacuole to the cytosol of an antigen-presenting cell.

22. A composition according to claim 19, wherein the cytolysin is a listeriolysin.

23. A method for stimulating an immune response in a subject that is naïve to a target antigen or that has previously raised an immune response to the target antigen, the method comprising administering to the subject an effective amount of an immune stimulator that stimulates or otherwise enhances an immune response to the target antigen and an inhibitor of IL-10 function.

24. A method according to claim 23, wherein the immune response is a T-cell mediated immune response.

25. A method according to claim 23, wherein the immune response is required for the treatment or prophylaxis of a disease or condition associated with the presence or aberrant expression of a target antigen in a subject.

26. A method according to claim 25, wherein the disease or condition is selected from a pathogenic infection, a disease characterized by immunodeficiency or a cancer.

27. A method according to claim 23, further comprising administering at least one other effective amount of the immune stimulator and optionally at least one other effective amount of the inhibitor of IL-10 function, to thereby maintain or enhance the immune response to the target antigen(s).

28. Use of an inhibitor of IL-10 function and an immune-stimulating agent that stimulates or otherwise enhances an immune response to a target antigen in the manufacture of a medicament for stimulating or enhancing an immune response to the target antigen.

29. Use of an inhibitor of IL-10 function and an immune-stimulating agent that stimulates or otherwise enhances an immune response to a target antigen in the manufacture of a medicament for treating or preventing a disease or condition associated with the presence or aberrant expression of the target antigen.
